(19) United States
(12) Patent Application Publication
(10) Pub. No.: US 2023/0365979 A1
(43) Pub. Date: Nov. 16, 2023

(54) PEPTIDE TAG AND TAGGED PROTEIN INCLUDING SAME

(71) Applicant: IDEMITSU KOSAN CO.,LTD., Chiyoda-ku (JP)

(72) Inventors: Kazuyoshi KOIKE, Chiyoda-ku (JP); Seika OIWA, Chiyoda-ku (JP)

(73) Assignee: IDEMITSU KOSAN CO.,LTD., Chiyoda-ku (JP)

(21) Appl. No.: 18/246,917

(22) PCT Filed: Sep. 28, 2021
(86) PCT No.: PCT/JP2021/035568

§ 371 Date: Mar. 28, 2023

(30) Foreign Application Priority Data
Sep. 29, 2020 (JP) 2020-163476

(51) Int. Cl. C12N 15/62; C12P 21/02; C12N 15/70; C12N 15/81; C07K 14/00

(52) U.S. Cl. CPC C12N 15/62; C12P 21/02; C12N 15/70; C12N 15/815; C07K 14/00; C07K 2319/00

(57) ABSTRACT

A peptide of 6 to 50 amino acid residues comprising the following sequence:

Xₘ(JYₙ)_{q}JZᵣ (I)
wherein J is an amino acid residue selected from Q (glutamine), E (glutamic acid), and G (glycine);
X and Y are each an amino acid residue independently selected from arginine (R), glycine (G), serine (S), lysine (K), threonine (T), leucine (L), asparagine (N), glutamine(Q), histidine (H), proline (P), isoleucine (I), valine (V), alanine (A), and methionine (M) with the proviso that X and Y are each other than Q in the case of said peptide containing Q as J and X and Y are each other than G in the case of said peptide containing G as J, and
at least one Y in each repeating unit JYₙ is K, L, N, Q, H or R;
Z is an amino acid residue independently selected from R, G, S, K, T, N, Q, H and P with the proviso that Z is other than Q in the case of said peptide containing Q as J and Z is other than G in the case of said peptide containing G as J;
the number of P's contained in the peptide is 0 or 1; and
m is an integer of 0 to 6, n is 1, 2 or 3, q is an integer of 1 to 10, and r is an integer of 0 to 10.

### TECHNICAL FIELD

The present invention relates to a peptide tag, and a tagged protein comprising the same, a DNA encoding the same, a transformant comprising the DNA, as well as a method of producing a tagged protein.

### BACKGROUND ART

According to the advancement of the gene recombination technique, production of useful proteins by heterologous expression is commonly performed these days. Solutions studied for improvements in expression of proteins and their amounts accumulated, in production of useful proteins by heterologous expression, are selection of promotors and terminators, translational enhancers, codon modification of transgenes, intracellular transport and localization of proteins, and the like. For example, Patent Document 1 discloses a technique for expressing a bacterial toxin protein in a plant or the like, and discloses expression of a bacterial toxin protein by linking with a peptide linker where prolines are arranged at certain intervals (Patent Document 1).

Furthermore, there have been developed several techniques where a peptide tag is linked to a protein of interest to result in an improvement in expression thereof (Patent Documents 2 to 6 and Non-Patent Documents 1 to 3).

### PRIOR ART DOCUMENTS

### Patent Documents

Patent Document 1: JP 5360727 B
Patent Document 2: JP 5273438 B
Patent Document 3: International Publication WO2016/204198
Patent Document 4: International Publication WO2017/115853
Patent Document 5: International Publication WO2020/045530
Patent Document 6: US 20090137004 A

### Non-Patent Documents

Non-Patent Document 1: Smith, D. B. and Johnson, K. S.: Gene, 67, 31, 1988
Non-Patent Document 2: Marblestone, J. G. et al.: Protein Sci., 15, 182, 2006
Non-Patent Document 3: di Guan, C. et al.: Gene, 67, 21, 1988, SUMMARY OF THE INVENTION

### Problems to be Solved by the Invention

The peptide linker where prolines are arranged at certain intervals, as disclosed in Patent Document 1, has been used to link a toxin fusion protein, thereby allowing for an increase in accumulation of a toxin fusion protein in a plant. Patent Documents 4 and 5 have each studied an amino acid between prolines in a peptide tag to thereby provide a peptide tag suitable for high expression and soluble expression of a protein. However, such peptide linker and peptide tag are on the condition that prolines arranged at certain intervals are present, and there is room for further studies of sequences in order to improve performances of tags for high protein expression. Accordingly, an object of the present invention is to provide a new peptide tag capable of linking to a protein of interest and thus increasing the expression level of the protein of interest in the case of expression of the protein of interest in a host cell or a cell-free expression system.

### Means for Solving the Problems

The present inventors have made studies about sequences in order to improve performances of the peptide tags disclosed in Patent Documents 4 and 5. The present inventors have then surprisingly found that, when a peptide tag having a sequence, where prolines arranged at certain intervals are each replaced with glutamine, glutamic acid, or glycine, is used to investigate the expression level of a protein to which the tag is added, the expression level of such a protein of interest is remarkably improved. The present invention has been made based on such findings.

The present invention provides the followings:
[1] A peptide of 6 to 50 amino acid residues comprising the following sequence:

Xₘ(JYₙ)_{q}JZᵣ (I)
wherein J is an amino acid residue selected from Q (glutamine), E (glutamic acid), and G (glycine);
X and Y are each an amino acid residue independently selected from arginine (R), glycine (G), serine (S), lysine (K), threonine (T), leucine (L), asparagine (N), glutamine(Q), histidine (H), proline (P), isoleucine (1), valine (V), alanine (A), and methionine (M) with the proviso that X and Y are each other than Q in the case of said peptide containing Q as J and X and Y are each other than G in the case of said peptide containing G as J, and
at least one Y in each repeating unit JYₙ is K, L, N, Q, H or R;
Z is an amino acid residue independently selected from R, G, S, K, T, N, Q, H and P with the proviso that Z is other than Q in the case of said peptide containing Q as J and Z is other than G in the case of said peptide containing G as J;
the number of P's contained in the peptide is 0 or 1; and
m is an integer of 0 to 6, n is 1, 2 or 3, q is an integer of 1 to 10, and r is an integer of 0 to 10.
[2] The peptide according to [1], comprising the sequence selected from the following (1) to (3):
   (1) Xₘ(QYₙ)_{q}QZᵣ
   (2) Xₘ(EYₙ)_{q}EZᵣ
   (3) Xₘ(GYₙ)_{q}GZᵣ
   in (1), X and Y are each an amino acid residue independently selected from R, G, S, K, T, L, N, H and P and at least one Y contains K, L, N, H or R, and Z is an amino acid residue independently selected from R, G, S, K, T, N, H and P;
   in (2), X and Y are each an amino acid residue independently selected from R, G, S, K, T, L, N, Q, H and P and at least one Y contains K, L, N, Q, H or R, and Z is an amino acid residue independently selected from R, G, S, K, T, N, Q, H and P; and
   in (3), X and Y are each an amino acid residue independently selected from R, S, K, T, L, N, Q, H and P and at least one Y contains K, L, N, Q, H or R, and Z is an amino acid residue independently selected from R, S, K, T, N, Q, H and P.
[3] The peptide according to [2], wherein
   in (1), X and Y are each an amino acid residue independently selected from R, K and N and at least one Y contains R, K or N,
   in (2), X and Y are each an amino acid residue independently selected from R, K, N and Q and at least one Y contains R, K, N or Q, and
   in (3), X and Y are each an amino acid residue independently selected from R, K, N and Q and at least one Y contains R, K, N or Q.
[4] The peptide according to [3], wherein
   in (1), Xₘ is (R/G/S/I/V/T/N/H/P/A/M)(K/N)(K/N), Yₙ is (K/N)(K/N), and Zᵣ is RS, NKPRS (SEQ ID NO:45) or KNPRS (SEQ ID NO:46),
   in (2), Xₘ is ((R/G/S/I/V/T/N/H/P/A/M)(K/N/Q)(K/N), Yₙ is (K/N/Q)(K/N), and Zᵣ is RS, KNPRS (SEQ ID NO:46) or QNPRS (SEQ ID NO:64), and
   in (3), Xₘ is (R/S/I/V/T/N/H/P/A/M)(K/N/Q)(K/N), Yₙ is (K/N/Q)(K/N), and Zᵣ is RS, NKPRS (SEQ ID NO:45) or KNPRS (SEQ ID NO:46).
[5] The peptide according to any of [1] to [3], wherein n is 2 or 3.
[6] The peptide according to any of [1] to [5], wherein q is an integer of 2 to 5.
[7] The peptide according to any of [1] to [6], comprising the amino acid sequence selected from SEQ ID NOs:1 to 4 and SEQ ID NOs:47 to 62.
[8] A tagged protein comprising the peptide according to any of [1] to [7] and a useful protein.
[9] The tagged protein according to [8], wherein the useful protein is an enzyme, a cytokine, an antibody, or a fluorescent protein.
[10] A DNA encoding the tagged protein according to [8] or [9].
[11] A recombinant vector comprising the DNA according to [10].
[12] A transformant transformed with the DNA according to [10] or the recombinant vector according to [11].
[13] A method of producing a tagged protein, comprising culturing the transformant according to [12] and expressing and accumulating a tagged protein, and recovering the tagged protein.
[14] A method of producing a tagged protein, comprising introducing the DNA according to [10] or an RNA transferred therefrom into a cell-free expression system and expressing and accumulating a tagged protein, and recovering the tagged protein.

### Advantageous Effects of the Invention

The peptide tag of the present invention can be used to thereby improve the expression level of a protein of interest. Accordingly, the peptide tag is useful for production of a protein with a host cell such as yeast, E. coli or Brevibacillus, or a cell-free expression system.

### BRIEF DESCRIPTION OF THE DRAWINGS

FIG. 1 A schematic diagram of the tagged protein expression vector (tagged at the N-terminal side) constructs for E. coli and cell-free use.

FIG. 2 A schematic diagram of the tagged protein expression vector (tagged at the C-terminal side) constructs for E. coli and cell-free use.

FIG. 3 A schematic diagram of an E. coli-Yarrowia lipolytica shuttle vector.

FIG. 4 A schematic diagram of the tagged protein expression vector construct for Yarrowia lipolytica.

FIG. 5 A graph illustrating the expression level of the tagged green fluorescent protein (GFP2) in a cell-free expression system. The graph illustrates a relative value under the assumption that the expression level of non-tagged GFP2 (Comparative Example A) is 1.

FIG. 6 A graph illustrating the expression level of the tagged VHH antibody in a cell-free expression system. The graph illustrates a relative value under the assumption that the expression level of a non-tagged VHH antibody (Comparative Example A) is 1.

FIG. 7 A graph illustrating the expression level of the tagged xylanase (XynA) in a cell-free expression system. The graph illustrates a relative value under the assumption that the expression level of non-tagged XynA (Comparative Example A) is 1.

FIG. 8 A graph illustrating the TPTG-induced expression level of the tagged GFP2 in E. coli (BL21). The graph illustrates a relative value under the assumption that the expression level of non-tagged GFP2 is 1.

FIG. 9 A graph illustrating the fluorescence intensity of the tagged GFP2 expressed under IPTG induction in E. coli (BL21). The graph illustrates a relative value under the assumption that the fluorescence intensity of non-tagged GFP2 is 1.

FIG. 10 A graph illustrating the IPTG induced-expression level of GFP2 tagged at the N-terminal side, in E. coli (BL21). The graph illustrates a relative value under the assumption that expression level of non-tagged GFP2 is 1.

FIG. 11 A graph illustrating the fluorescence intensity of the GFP2 tagged at the N-terminal side, expressed under IPTG induction in E. coli (BL21). The graph illustrates a relative value under the assumption that the fluorescence intensity of non-tagged GFP2 is 1.

FIG. 12 A graph illustrating the IPTG induced-expression level of GFP2 tagged at the C-terminal side, in E. coli (BL21). The graph illustrates a relative value under the assumption that expression level of non-tagged GFP2 is 1.

FIG. 13 A graph illustrating the fluorescence intensity of the GFP2 tagged at the C-terminal side, expressed under IPTG induction in E. coli (BL21). The graph illustrates a relative value under the assumption that the fluorescence intensity of non-tagged GFP2 is 1.

FIG. 14 A graph illustrating the IPTG induced-expression level of GFP2 tagged at the N-terminal side, in Yarrowia lipolytica. The graph illustrates a relative value under the assumption that expression level of non-tagged GFP2 is 1.

### EMBODIMENTS FOR CARRYING OUT THE INVENTION

The peptide of the present invention (also referred to as “peptide tag”) has the following amino acid sequence.

Xₘ(JYₙ)_{q}JZᵣ (I)

Herein, J is an amino acid selected from Q (glutamine), E (glutamic acid) and G (glycine). J contained in the peptide of the present invention may be 2 or 3 kinds of amino acid residues selected from Q, E and G, but is preferably one kind of amino acid residue selected from Q, E and G.

Accordingly, preferable aspects of the peptide of the present invention include respective peptides of (1) to (3) described below.

X is an amino acid residue independently selected from arginine (R), glycine (G), serine (S), lysine (K), threonine (T), leucine (L), asparagine (N), glutamine(Q), histidine (H), proline (P), isoleucine (I), valine (V), alanine (A) and methionine (M), preferably an amino acid residue independently selected from R, K, N, Q, G, S, I, V, T, N, H, P, A and M. X is an amino acid other than Q selected from said amino acid residues in the case of the sequence (I) containing Q as J and X is an amino acid other than G selected from said amino acid residues in the case of the sequence (I) containing G as J.

Xₘ means in-consecutive X's, and m-consecutive X's may be rn-consecutive same kind of amino acid residue or different kinds of amino acid residues selected from R, G, S, K, T, L, N, Q, H, P, I, V, A, and M. m is an integer of 0 to 6, and is preferably an integer of 0 to 5, more preferably an integer of 1 to 5, further preferably an integer of 1 to 3.

Xₘ is, for example, R(K/N/Q)(K/N).

Xₘ is, for example, (R/G/S/I/V/T/N/H/P/A/M)(K/N/Q)(K/N). This sequence may be repeated twice.

Xₘ is more preferably (R/G/S/I/V/T/N/H/P/A/M)(K/N)(K/N). This sequence may be repeated twice.

Xₘ is further preferably (R/G/S/I/V/T/N/H/P/A/M)KN, (R/G/S/I/V/T/N/H/P/A/M)NK. This sequence may be repeated twice.

Xₘ is, for example, RQN or RQNPQN (SEQ ID NO:63).

Y is an amino acid residue independently selected from R, G, S, K, T, L, N, Q, H, and P, preferably an amino acid residue independently selected from R, K, N, and Q. Y is an amino acid other than Q selected from said amino acid residues in the case of the sequence (I) containing Q as J in and Y is an amino acid other than G selected from said amino acid residues in the case of the sequence (I) containing G as J.

(JYₙ)_{q} means that JYₙ, n being 1, 2 or 3, JY, JYY or JYYY is continued q times (J represents Q, E or G). JY, JYY and/or JYYY may be continued q times in total.

Such Y's may be here either the same kind of amino acid residue or different kinds of amino acid residues selected from R, G, S, K, T, L, N, Q, H, and P, and, preferably, at least one of such Y's contained in each repeating unit JYₙ represents K, L, N, Q, H or R and at least one thereof represents K, N, Q or R. More preferably, two or more of such Y's contained in such each JYₙ represent K, L, N, Q, H or R, and further preferably, two or more of such Y's contained in such each JYₙ represent K, N, Q or R. Herein, n is preferably 2 or 3, more preferably 2. q is an integer of 1 to 10, preferably an integer of 2 to 10, more preferably an integer of 2 to 5, further preferably an integer of 2 to 3.

JYₙ is, for example, J(K/N/Q)(K/N).

JYₙ is, for example, J(K/N)(K/N).

JYₙ is preferably JKN or JNK.

When J is E, JYₙ may be JQN.

Z is an amino acid residue independently selected from R, G, S, K, T, N, Q, H and P, preferably an amino acid residue independently selected from R and S. Z is an amino acid other than Q selected from said amino acid residues in the case of the sequence (I) containing Q as J and Z is an amino acid other than G selected from said amino acid residues in the case of the sequence (I) containing G as J.

JZᵣ means r-consecutive Z's following J, and r-consecutive Z's may be either the same kind of amino acid residue or different kinds of amino acid residues selected from R, G, S, K, T, N, Q, and P. r is an integer of 0 to 10, and is preferably an integer of 1 to 10, more preferably an integer of 1 to 5.

JZᵣ is, for example, JRS, and may be NKPRS (SEQ ID NO:45), KNPRS (SEQ ID NO:46) or QNPRS (SEQ ID NO:64).

The number of P's contained in the peptide of the present invention is 0 or 1. Accordingly, Y and Z contain no P in the case that one P is contained as X, X and Z contain no P in the case that one P is contained as Y, and X and Y contain no P in the case that one P is contained as Z. The same applies to the following peptides (1) to (3).

The peptide of the present invention has a length of preferably 6 to 50 amino acids, more preferably 6 to 40 amino acids, further preferably 8 to 40 amino acids, still preferably 10 to 30 amino acids, still more preferably 10 to 25 amino acids, particularly preferably 12 to 20 amino acids.

Preferable examples of the peptide of the present invention include the following (1) to (3) where J contained in each of the peptides is any one of G, E or Q.
(1) Xₘ(QYₙ)_{q}QZᵣ
(2) Xₘ(EYₙ)_{q}EZᵣ
(3) Xₘ(GYₙ)_{q}GZᵣ

In (1), X and Y are each an amino acid residue independently selected from R, G, S, K, T, L, N, H, P, I, V, A, and M, and, preferably, X is an amino acid residue independently selected from R, K, N, G, S, I, V, T, N, H, P, A, M and Y is an amino acid residue independently selected from R, K, and N.

At least one Y contained in each repeating unit QYₙ contains K, L, N, H or R, and at least one therein preferably contains K, N or R.

Z is an amino acid residue independently selected from R, G, S, K, T, N, H, and P.

In (1), m, n, q, and r are numbers defined as in m, n, q, and r in (I), and respective preferred numerical ranges thereof are also defined in the same manner. Accordingly, Xₘ is the same as Xₘ described with respect to (I) except that X contains no Q, (QYₙ)_{q} is the same as (JYₙ)_{q} where J is replaced by Q, described with respect to (I), except that Y contains no Q, and QZᵣ is the same as JZᵣ where J is replaced by Q, described with respect to (I), except that Z contains no Q.

Xₘ is, for example, (R/G/S/I/V/T/N/H/P/A/M)(K/N)(K/N). This sequence may be repeated twice.

Xₘ is further preferably (R/G/S/I/V/T/N/H/P/A/M)KN, (R/G/S/I/V/T/N/H/P/A/M)NK. This sequence may be repeated twice.

Yₙ is, for example, (K/N)(K/N).

Yₙ is preferably KN or NK.

Zᵣ is, for example, RS, and may be NKPRS (SEQ ID NO:45) or KNPRS (SEQ ID NO:46).

In (2), X and Y are each an amino acid residue independently selected from R, G, S, K, T, L, N, Q, H, P, I, V, A, and M, and, preferably, X is an amino acid residue independently selected from R, K, N, Q, G, S, I, V, T, N, H, P, A and M and Y is an amino acid residue independently selected from R, K, N, and Q.

At least one Y contained in each repeating unit EYₙ contains K, L, N, Q, H or R, and at least one therein preferably contains K, N, Q or R.

Z is an amino acid residue independently selected from R, G, S, K, T, N, Q, H and P.

In (2), m, n, q, and r are numbers defined as in m, n, q, and r in (I), and respective preferred numerical ranges thereof are also defined in the same manner. Accordingly, Xₘ is the same as Xₘ described with respect to (1), (EYₙ)_{q} is the same as(JYₙ)_{q} where J is replaced by E, described with respect to (I), and EZᵣ is the same as JZᵣ where J is replaced by E, described with respect to (I).

Xₘ is, for example, (R/G/S/I/V/T/N/H/P/A/M)(K/N/Q)(K/N). This sequence may be repeated twice.

Xₘ is further preferably (R/G/S/I/V/T/N/H/P/A/M)KN, (R/G/S/I/V/T/N/H/P/A/M)QN. This sequence may be repeated twice.

Yₙ is, for example, (K/N/Q)(K/N).

Yₙ is preferably KN or QN.

Zᵣ is, for example, RS, and may be KNPRS (SEQ ID NO:46) or QNPRS (SEQ ID NO:64).

In (3), X and Y are each an amino acid residue independently selected from R, S, K, T, L, N, Q, H, P, I, V, A, and M, and, preferably, X is an amino acid residue independently selected from R, K, N, Q, G, S, I, V, T, N, H, P, A and M and Y is an amino acid residue independently selected from R, K, N, and Q.

At least one Y contained in each repeating unit GYₙ contains K, L, N, Q, H or R, and at least one therein preferably contains K, N, Q or R. Z is an amino acid residue independently selected from R, S. K, T, N, Q, H, and P.

In (1), m, n, q, and r are numbers defined as in m, n, q, and r in (I), and respective preferred numerical ranges thereof are also defined in the same manner. Accordingly, Xₘ is the same as Xₘ described with respect to (I) except that X contains no G, (GYₙ)_{q} is the same as (JYₙ)_{q} where J is replaced by G, described with respect to (I), except that Y contains no G, and GZᵣ is the same as JZᵣ where J is replaced by G, described with respect to (I), except that Z contains no G.

Xₘ is, for example, (R/S/1/V/T/N/H/P/A/M)(K/N/Q)(K/N). This sequence may be repeated twice.

Xₘ is further preferably (R/G/S/I/V/T/N/H/P/A/M)KN, (R/G/S/I/V/T/N/H/P/A/M)NK. This sequence may be repeated twice.

Yₙ is, for example, (K/N/Q)(K/N).

Yₙ is preferably KN or NK.

Zᵣ is, for example, RS, and may be NKPRS (SEQ ID NO:45) or KNPRS (SEQ ID NO:46).

Specific examples of the peptide of the present invention include, but not limited to, a peptide having the amino acid sequence selected from SEQ ID NOs:1 to 4 and 47 to 62.

(SEQ ID NO: 1)
RKNGKNGKNGRS
(SEQ ID NO: 2)
RKNEKNEKNERS
(SEQ ID NO: 3)
RNKQNKQNKQRS
(SEQ ID NO: 4)
RQNEQNEQNERS
(SEQ ID NO: 47)
RNKPNKQNKQRS
(SEQ ID NO: 48)
RNKQNKQNKPRS
(SEQ ID NO: 49)
RQNPQNEQNERS
(SEQ ID NO: 50)
RNKQNKQRS
(SEQ ID NO: 51)
GNKQNKQNKQRS
(SEQ ID NO: 52)
SNKQNKQNKQRS
(SEQ ID NO: 53)
INKQNKQNKQRS
(SEQ ID NO: 54)
VNKQNKQNKQRS
(SEQ ID NO: 55)
TNKQNKQNKQRS
(SEQ ID NO: 56)
NNKQNKQNKQRS
(SEQ ID NO: 57)
HNKQNKQNKQRS
(SEQ ID NO: 58)
PNKQNKQNKQRS
(SEQ ID NO: 59)
ANKQNKQNKQRS
(SEQ ID NO: 60)
MNKQNKQNKQRS
(SEQ ID NO: 61)
RNKQNKQNKQNKQRS
(SEQ ID NO: 62)
RNKQNKQNKQNKQNKQRS

The tagged protein of the present invention is one in which the peptide tag of the present invention is linked to a protein of interest (also referred to as “fusion protein of a tag and a protein of interest”). The peptide tag may be linked to the N-terminus of a protein of interest, the peptide tag may be linked to the C-terminus of a protein of interest, or the peptide tag may be linked to both the N-terminus and the C-terminus of a protein of interest. The peptide tag may be linked directly or through a sequence of one to several amino acids (for example, 1 to 5 amino acids), to the N-terminus and/or the C-terminus of a protein of interest. The sequence of one to several amino acids may be any sequence as long as it is a sequence having no adverse effect on the function and the expression level of the tagged protein, and can be a protease recognition sequence to thereby allow the peptide tag to be cleaved off from a useful protein after expression and purification. Examples of the protease recognition sequence include a factor Xa recognition sequence. The tagged protein of the present invention may also include any other tag sequence required for detection, purification, and/or the like, such as a His tag, an HN tag, or a FLAG tag.

Examples of the useful protein contained in the tagged protein of the present invention include, but not limited to, growth factors, hormones, cytokines, blood proteins, enzymes, antigens, antibodies, transcription factors, receptors, fluorescent proteins, and partial peptides thereof.

Examples of the enzymes include lipase, protease, steroid-synthesizing enzymes, kinase, phosphatase, xylanase, esterase, methylase, demethylase, oxidase, reductase, cellulase, aromatase, collagenase, transglutaminase, glycosidase, and chitinase.

Examples of the growth factors include epidermal growth factor (EGF), insulin-like growth factor (IGF), transforming growth factor (TGF), nerve growth factor (NGF), brain-derived neurotrophic factor (BDNF), vascular endothelial growth factor (VEGF), granulocyte colony-stimulating factor (G-CSF), granulocyte-macrophage colony-stimulating factor (GM-CSF), platelet-derived growth factor (PDGF), erythropoietin (EPO), thrombopoietin (TPO), fibroblast growth factor (FGF), and hepatocyte growth factor (HGF).

Examples of the hormones include insulin, glucagon, somatostatin, growth hormone, parathyroid hormone, prolactin, leptin, and calcitonin.

Examples of the cytokines include interleukins, interferons (IFNα, TFNβ, IFNγ), and tumor necrosis factor (TNF).

Examples of the blood proteins include thrombin, serum albumin, factor VII, factor VIII, factor IX, factor X, and tissue plasminogen activator.

Examples of the antibodies include complete antibodies, Fab, F(ab′), F(ab′)₂, FEc, Fc fusion proteins, heavy chain (H-chain), light chain (L-chain), single-chain Fv (scFv), sc(Fv)₂, disulfide-linked Fv (sdFv), Diabodies, and VHH antibodies.

The antigen proteins for use as vaccines are not particularly limited as long as these can induce the immune response, and may be appropriately selected depending on the expected target of the immune response, and examples thereof include proteins derived from pathogenic bacteria and proteins derived from pathogenic viruses.

A secretion signal peptide which functions in a host cell may be added for secretory production, to the tagged protein of the present invention. Examples of the secretion signal peptide include invertase secretion signal, P3 secretion signal, and a factor secretion signal in the case of yeast as the host, PelB secretion signal in the case of E. coli as the host, and P22 secretion signal in the case of Brevibacillus as the host. In the case of a plant as the host, examples include secretion signal derived from a plant belonging to the nightshade family (Solanaceae), the rose family (Rosaceae), the mustard family (Brassicaceae), or the composite family (Asleraceae), further preferably a plant belonging to the genus Nicoliana, the genus Arabidopsis, the genus Fragaria, the genus Lactuca, or the like, preferably tobacco (Nicotiana tabacum), Arabidopsis thaliana, strawberry (Fragaria x ananassa), lettuce (Lactuca sativa), or the like.

A transport signal peptide such as an endoplasmic reticulum retention signal peptide or a vacuole transport signal peptide may be further added to the tagged protein of the present invention in order to allow for expression in a particular cellular compartment.

The tagged protein of the present invention can be chemically synthesized, or can be produced by genetic engineering. The method for production by genetic engineering is described below.

The DNA of the present invention comprises a DNA encoding the tagged protein of the present invention. In other words, the DNA of the present invention comprises a DNA encoding the useful protein and a DNA encoding the peptide tag. The DNA encoding the useful protein and the DNA encoding the peptide tag are linked in reading frame.

The DNA encoding the useful protein can be obtained by, for example, a common genetic engineering procedure based on a known base sequence.

In the DNA encoding the tagged protein of the present invention, a codon encoding an amino acid constituting the tagged protein is preferably also appropriately modified so that the translational level of a fusion protein is increased depending on the host cell which produces the protein. Examples include a method in which a codon high in frequency of use in the host cell is selected, a method in which a codon high in GC content is selected, and a method in which a codon high in frequency of use in a housekeeping gene of the host cell is selected.

The DNA of the present invention may contain an enhancer sequence or the like which functions in the host cell, in order to improve expression in the host cell. Examples of the enhancer include a Kozak sequence and a 5′-untranslated region of an alcohol dehydrogenase gene derived from a plant.

The DNA of the present invention can be produced by a common genetic engineering procedure, and can be constructed by, for example, linking, for example, the DNA encoding the peptide tag of the present invention and the DNA encoding the useful protein with PCR, DNA ligase, or the like.

The recombinant vector of the present invention may be one in which the DNA encoding the tagged protein is inserted into a vector so that the DNA can be expressed in the host cell into which the vector is to be introduced. The vector is not particularly limited as long as it can replicate in the host cell, and examples thereof include plasmid DNA and viral DNA. The vector preferably contains a selection marker such as a drug resistance gene. Specific examples of the plasmid vector include pTrcHis2 vector, pUC119, pBR322, pBluescript II KS+, pYES2, pAUR123, pQE-Tri, pET, pGEM-3Z, pGEX, pMAL, pRI909, pRI910, pBI221, pBI121, pBI101, pIG121Hm, pTrc99A, pKK223, pA1-11, pXT1, pRc/CMV, pRc/RSV, pcDNA I/Neo, p3×FLAG-CMV-14, pCAT3, pcDNA3.1, and pCMV.

The promotor for use in the vector can be appropriately selected depending on the host cell into which the vector is to be introduced. For example, in the case of expression in yeast, a GAL1 promotor, a PGK1 promotor, a TEF1 promotor, an ADH1 promotor, a TPI1 promotor, a PYK1 promotor, or the like can be used. In the case of expression in a plant, a cauliflower mosaic virus 35S promotor, a rice actin promotor, a maize ubiquitin promotor, a lettuce ubiquitin promotor, or the like can be used. In the case of expression in E. coli, examples include a T7 promotor, and in the case of expression in Brevibacillus, examples include a P2 promotor and a P22 promotor. An inducible promotor may be adopted, and examples of the inducible promotor which can be used include not only lac, tac, and trc as promotors which are inducible with IPTG, but also trp which is inducible with IAA, ara which is inducible with L-arabinose, Pzt-1 which is inducible with tetracycline, a P_{L} promotor which is inducible at high temperature (42° C.), and a promotor of a cspA gene which is one cold shock gene.

A terminator sequence can also be, if necessary, included depending on the host cell.

The recombinant vector of the present invention can be prepared by, for example, cleaving a DNA construct with an appropriate restriction enzyme, or adding a restriction enzyme site thereto by PCR, and then inserting the resultant into a restriction enzyme site or a multicloning site in a vector.

The transformant of the present invention is transformed with the DNA or the recombinant vector including it. The host cell for use in transformation may be any of a eukaryotic cell and a prokaryotic cell.

The eukaryotic cell preferably used is a yeast cell, a mammalian cell, a plant cell, an insect cell, or the like. Examples of the yeast include Saccharomyces cerevisiae, Candida utilis, Schizosaccharomyces pombe, Pichia pastoris, Yarrowia lipolytica, and Metschnikowia pulcherrima. Microorganisms such as Aspergillus can also be used. Examples of the prokaryotic cell include E. coli (Escherichia coli), Lactobacillus, Bacillus, Brevibacillus, Agrobacterium tumefaciens, Streptomyces, and Corynebacterium. Examples of the plant cell include cells of plants belonging to the composite family (Astaraceae) such as the genus Lactuca, the nightshade family (Solanaceae), the mustard family (Brassicaceae), the rose family (Rosaceae), the chenopodiaceous family (Chenopodiaceae), and the like.

The transformant for use in the present invention can be produced by introducing the recombinant vector of the present invention into the host cell by use of a common genetic engineering procedure. For example, a method can be used, for example, the electroporation method (Tada, et al., 1990, Theor. Appl. Genet, 80: 475), the protoplast method (Gene, 39, 281-286 (1985)), the polyethylene glycol method (Lazzeri, et al., 1991, Theor. Appl. Genet. 81:437), the introduction method utilizing Agrobacterium (Hood, et al., 1993, Transgenic, Res. 2: 218, Hiei, et al., 1994 Plant J. 6: 271), the particle gun method (Sanford, et al., 1987, J. Part. Sci. tech. 5:27), or the polycation method (Ohtsuki, et al., FEBS Lett. 1998 May 29; 428(3): 235-40.). The gene expression may be transient expression, or may be stable expression with incorporation into the chromosome.

The transformant can be selected with the phenotype of a selection marker after introduction of the recombinant vector of the present invention into the host cell. The tagged protein can be produced by culturing the transformant selected. The medium and conditions for use in the culturing can be appropriately selected depending on the type of the transformant.

In the case where the host cell is a plant cell, a plant body can be regenerated by culturing the plant cell selected, according to an ordinary method, and the tagged protein can be accumulated inside the plant cell or outside the membrane of the plant cell.

A protein to which the peptide tag of the present invention is added can be expressed also by introducing the DNA of the present invention, RNA transferred therefrom (mRNA), or the recombinant vector of the present invention, into the cell-free expression system.

The cell-free expression system is not particularly limited as long as it is an expression system including a protein expression mechanism such as ribosome, and may be a protein expression system obtained by reconstituting a cell extract such as an E. coli-derived cell extract, a wheat germ-derived cell extract, a rabbit reticulocyte-derived cell extract, or an insect cell-derived cell extract, or a factor such as ribosome.

A protein to which the peptide tag of the present invention is added, accumulated in a medium, in a cell, or in a cell-free expression system, can be separated and purified according to a method well known to those skilled in the art. For example, the separation and purification may be carried out by an appropriate known method such as salting-out, ethanol precipitation, ultrafiltration, gel filtration chromatography, ion-exchange column chromatography, affinity chromatography, high/medium-pressure liquid chromatography, reversed-phase chromatography, or hydrophobic chromatography, or by combination of any of these.

Hereinafter, Examples of the present invention are described, but the present invention is not limited to such Examples.

### EXAMPLES

### (1) Construction of Various Plasmids Encoding Various Tagged Proteins

Artificial synthetic DNAs (SEQ ID NOs:9, 11, 13) encoding various proteins (GFP2, VHH antibody, XynA) were each inserted into the EcoRV recognition site of the pUC19-modified plasmid pUCFa (Fasmac), to thereby obtain various plasmids 1 to 3.

A pET28a plasmid (Invitrogen) having a T7 promotor was used as a plasmid for E. coli and cell-free system expression, and each plasmid for expression of a fusion protein where various peptide tags were each added at the N-terminus or the C-terminus of each of various proteins was constructed by the following procedure.

First, PCR by the combination of a template plasmid, a forward primer, and a reverse primer shown in Table 2 and Table 3 was performed for addition of each of various peptide tags (Table 1) to the N-terminus of each of various proteins. A sequence homologous to the pET28a plasmid was added to the 5′-end of each primer. KOD-PLUS-Ver.2 (Toyobo Co., Ltd.) was used for the PCR, 50 μl of a reaction liquid was prepared so that 2 pg/μl of a template plasmid, 0.3 μM of a forward primer, 0.3 μM of a reverse primer, 0.2 mM of dNTPs, 1×Buffer for KOD-Plus-Ver.2, 1.5 mM of MgSO₄, and 0.02 U/μl of KOD-PLUS-Ver.2 were contained, and was heated at 94° C. for 5 minutes and then subjected to heat treatment at 98° C. for 10 seconds, at 60° C. for 30 seconds, and at 68° C. for 40 seconds by 30 cycles and finally heated at 68° C. for 5 minutes. The resulting amplification fragment was purified with a QIAquick PCR Purification Kit (Qiagen).

pET28a plasmid was digested with NcoI and HindIII, and then separated by electrophoresis using 1.0% SeaKem GTG Agarose, and extracted from the gel by use of a QIAquick Gel Extraction Kit (Qiagen), to thereby obtain plasmid 4.

One μl of plasmid 4 extracted at a content of about 50 ng, 1 μl of a purified PCR product and 1 μl of sterile distilled water were mixed, and adjusted so that the amount of a liquid was 3 μl, and then the mixture was mixed with 0.75 μl of 5× In-Fusion HD Enzyme Premix attached to In-Fusion HD Cloning Kit (TaKaRa), incubated at 50° C. for 15 minutes, and then left to stand on ice for 5 minutes.

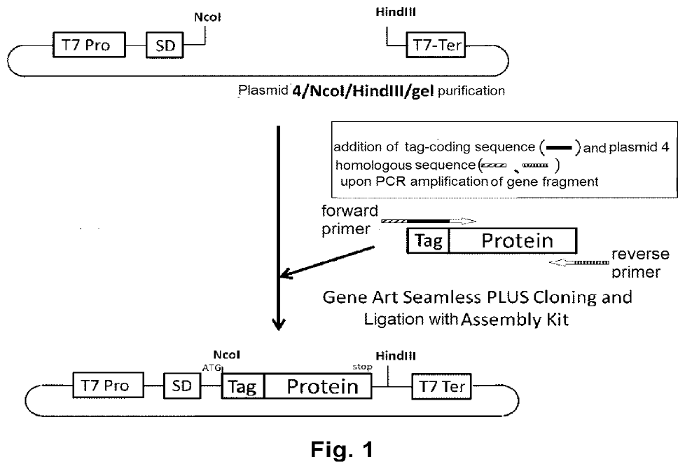

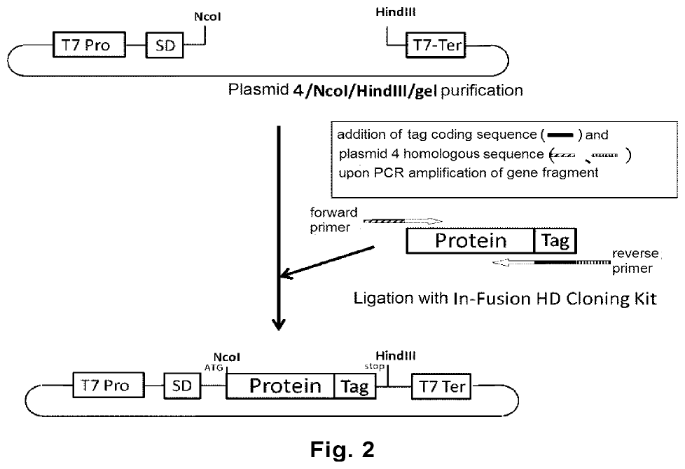

One μl of the reaction liquid was mixed with 15 μl of competent cells DH5-α, left to stand on ice for 30 minutes, then warmed at 42° C. for 45 seconds, and left to stand on ice for 2 minutes, thereafter 200 μl of SOC was added thereto, and the mixture was shaken at 37° C. and 200 rpm for 1 hour. Next, the entire amount of the shaken product was applied to 2×YT agar medium containing 100 mg/l kanamycin, and then subjected to static culture at 37° C. overnight, to thereby obtain a transformed colony. The colony was transferred to 4 ml of 2×YT liquid medium containing 100 mg/l kanamycin, and subjected to shake culture at 37° C. and 200 rpm overnight, thereafter a plasmid for gene expression, constructed by the procedure shown in FIG. 1 and FIG. 2, was extracted, the base sequence was confirmed, and thereafter the plasmid was used for an E. coli cell-free expression test and transformation of an E. coli (BL21 (DE3)) strain.

TABLE 1
Amino acid sequences of various tags

| | | |
|---|---|---|
| Example 1 | ZN12-B01 | RKNGKNGKNGRS |
| | | (SEQ ID NO: 1) |
| Example 2 | ZN12-B11 | RKNEKNEKNERS |
| | | (SEQ ID NO: 2) |
| Example 3 | ZN12-B15 | RNKQNKQNKQRS |
| | | (SEQ ID NO: 3) |
| Example 4 | ZN12-B19 | RQNEQNEQNERS |
| | | (SEQ ID NO: 4) |
| Example 5 | ZX12-B20 | RNKPNKQNKQRS |
| | | (SEQ ID NO: 4 7) |
| Example 7 | ZX12-B22 | RNKQNKQNKPRS |
| | | (SEQ ID NO: 4 8) |
| Example 8 | ZX12-B23 | RQNPQNEQNERS |
| | | (SEQ ID NO: 4 9) |
| Example 10 | ZX09-B15 | RNKQNKQRS |
| | | (SEQ ID NO: 5 0) |
| Example 1 1 | ZX12-B25 | GNKQNKQNKQRS |
| | | (SEQ ID NO: 5 1) |
| Example 1 2 | ZX12-B26 | SNKQNKQNKQRS |
| | | (SEQ ID NO: 5 2) |
| Example 1 3 | ZX12-B27 | INKQNKQNKQRS |
| | | (SEQ ID NO: 5 3) |
| Example 1 4 | ZX12-B28 | VNKQNKQNKQRS |
| | | (SEQ ID NO: 5 4) |
| Example 1 5 | ZX12-B29 | TNKQNKQNKQRS |
| | | (SEQ ID NO: 5 5) |
| Example 1 6 | ZX12-B30 | NNKQNKQNKQRS |
| | | (SEQ ID NO: 5 6) |
| Example 1 7 | ZX12-B31 | IINKQNKQNKQRS |
| | | (SEQ ID NO: 5 7) |
| Example 1 8 | ZX12-B32 | PNKQNKQNKQRS |
| | | (SEQ ID NO: 5 8) |
| Example 1 9 | ZX12-B33 | ANKQNKQNKQRS |
| | | (SEQ ID NO: 5 9) |
| Example 2 0 | ZX12-B35 | MNKQNKQNKQRS |
| | | (SEQ ID NO: 6 0) |
| Example 2 1 | ZX15-B15 | RNKQNKQNKQNKQRS |
| | | (SEQ ID NO: 6 1) |
| Example 2 2 | ZX18-B15 | RNKQNKQNKQNKQNKQRS |
| | | (SEQ ID NO: 6 2) |
| Comparative | No tag | |
| Example A | | |
| Comparative | PX12-20 | RKPGKGPGKPRS |
| Example B | | (SEQ ID NO: 1 5) |
| Comparative | PX12-20v7 | RKPKKKPKKPRS |
| Example C | | (SEQ ID NO: 1 6) |
| Comparative | PX12-90 | RQPQQQPQQPRS |
| Example D | | (SEQ ID NO: 1 7) |

The base sequences encoding SEQ ID NOs: 1 to 4 are respectively described by SEQ ID NOs:5 to 8, and the base sequences encoding SEQ ID NOs:15 to 17 are respectively described by SEQ ID NOs:18 to 20.

TABLE 2
Combination of template plasmid and primer used
in PCR amplification of each of various genes

| Forward Primer | Reverse Primer | Template Plasmid |
|---|---|---|
| GFP2-Nu11F(SEQ ID NO: 21) | GFP2-stopT7(SEQ ID NO: 29) | pUCFa-GFP2 |
| GFP2-ZX12-B01NF(SEQ ID NO: 22) | GFP2-stopT7(SEQ ID NO: 29) | pUCFa-GFP2 |
| GFP2-ZX12-B11NF(SEQ ID NO: 23) | GFP2-stopT7(SEQ ID NO: 29) | pUCFa-GFP2 |
| GFP2-ZX12-B15NF(SEQ ID NO: 24) | GFP2-stopT7(SEQ ID NO: 29) | pUCFa-GFP2 |
| GFP2-ZX12-B19NF(SEQ ID NO: 25) | GFP2-stopT7(SEQ ID NO: 29) | pUCFa-GFP2 |
| GFP2-PX12-20NF(SEQ ID NO: 26) | GFP2-stopT7(SEQ ID NO: 29) | pUCFa-GFP2 |
| GFP2-PX12-20v7NF(SEQ ID NO: 27) | GFP2-stopT7(SEQ ID NO: 29) | pUCFa-GFP2 |
| GFP2-PX12-90NF(SEQ ID NO: 28) | GFP2-stopT7(SEQ ID NO: 29) | pUCFa-GFP2 |
| VHH-Nu11F(SEQ ID NO: 30) | VHH-stopT7(SEQ ID NO: 36) | pUCFa-VHH |
| VHH-ZX12-01NF(SEQ ID NO: 31) | VHH-stopT7(SEQ ID NO: 36) | pUCFa-VHH |
| VHH-ZX12-11NF(SEQ ID NO: 32) | VHH-stopT7(SEQ ID NO: 36) | pUCFa-VHH |
| VHH-ZX12-15NF(SEQ ID NO: 33) | VHH-stopT7(SEQ ID NO: 36) | pUCFa-VHH |
| VHH-PX12-20NF(SEQ ID NO: 34) | VHH-stopT7(SEQ ID NO: 36) | pUCFa-VHH |
| VHH-PX12-20v7NF(SEQ ID NO: 35) | VHH-stopT7(SEQ ID NO: 36) | pUCFa-VHH |
| XynA-Nu11F(SEQ ID NO: 37) | XynA-stopT7(SEQ ID NO: 44) | pUCFa-VHH |
| XynA-ZX12-01NF(SEQ ID NO: 38) | XynA-stopT7(SEQ ID NO: 44) | pUCFa-XynA |
| XynA-ZX12-11NF(SEQ ID NO: 39) | XynA-stopT7(SEQ ID NO: 44) | pUCFa-XynA |
| XynA-ZX12-15NF(SEQ ID NO: 40) | XynA-stopT7(SEQ ID NO: 44) | pUCFa-XynA |
| XynA-PX12-20NF(SEQ ID NO: 41) | XynA-stopT7(SEQ ID NO: 44) | pUCFa-XynA |
| XynA-PX12-20v7NF(SEQ ID NO: 12) | XynA-stopT7(SEQ ID NO: 44) | pUCFa-XynA |
| XynA-PX12-90NF(SEQ ID NO: 43) | XynA-stopT7(SEQ ID NO: 44) | pUCFa-XynA |

TABLE 3
Combination of template plasmid and primer used in PCR
amplification of GFP2 gene for E. coli expression

| Forward Primer | Reverse Primer | Template Plasmid |
|---|---|---|
| GFP2-ZX12-B20NF(SEQ ID NO: 65) | GFP2-StopT7(SEQ ID NO: 29) | pUCFa-GFP2 |
| GFP2-ZX12-B22NT(SEQ ID NO: 66) | GFP2-StopT7(SEQ ID NO: 29) | pUCFa-GFP2 |
| GFP2-ZX12-B23NF(SEQ ID NO: 67) | GFP2-StopT7(SEQ ID NO: 29) | pUCFa-GFP2 |
| GFP2-ZX09-B15NF(SEQ ID NO: 68) | GFP2-StopT7(SEQ ID NO: 29) | pUCFa-GFP2 |
| GFP2-ZX12-B25NF(SEQ ID NO: 69) | GFP2-StopT7(SEQ ID NO: 29) | pUCFa-GFP2 |
| GFP2-ZX12-B26NF(SEQ ID NO: 70) | GFP2-StopT7(SEQ ID NO: 29) | pUCFa-GFP2 |
| GFP2-ZX12-B27NF(SEQ ID NO: 71) | GFP2-StopT7(SEQ ID NO: 29) | pUCFa-GFP2 |
| GFP2-ZX12-B28NF(SEQ ID NO: 72) | GFP2-StopT7(SEQ ID NO: 29) | pUCFa-GFP2 |
| GFP2-ZX12-B29NF(SEQ ID NO: 73) | GFP2-StopT7(SEQ ID NO: 29) | pUCFa-GFP2 |
| GFP2-ZX12-B30NF(SEQ ID NO: 74) | GFP2-StopT7(SEQ ID NO: 29) | pUCFa-GFP2 |
| GFP2-ZX12-B31NF(SEQ ID NO: 75) | GFP2-StopT7(SEQ ID NO: 29) | pUCFa-GFP2 |
| GFP2-ZX12-B32NF(SEQ ID NO: 76) | GFP2-StopT7(SEQ ID NO: 29) | pUCFa-GFP2 |
| GFP2-ZX12-B33NF(SEQ ID NO: 77) | GFP2-StopT7(SEQ ID NO: 29) | pUCFa-GFP2 |
| GFP2-ZX12-B35NF(SEQ ID NO: 78) | GFP2-StopT7(SEQ ID NO: 29) | pUCFa-GFP2 |
| GFP2-ZX15-B15NF(SEQ ID NO: 79) | GFP2-StopT7(SEQ ID NO: 29) | pUCFa-GFP2 |
| GFP2-ZX18-B15NF(SEQ ID NO: 80) | GFP2-StopT7(SEQ ID NO: 29) | pUCFa-GFP2 |
| GFP2-Nu11F(SEQ ID NO: 21) | GFP2-ZX12-B15C-Stopt7(SEQ ID NO: 81) | pUCFa-GFP2 |
| GFP2-Nu11F(SEQ ID NO: 21) | GFP2-ZX12-B26C-Stopt7(SEQ ID NO: 82) | pUCFa-GFP2 |
| GFP2-Nu11F(SEQ ID NO: 21) | GFP2-ZX12-B27C-Stopt7(SEQ ID NO: 83) | pUCFa-GFP2 |
| GFP2-Nu11F(SEQ ID NO: 21) | GFP2-ZX12-B33C-Stopt7(SEQ ID NO: 84) | pUCFa-GFP2 |
| GFP2-Nu11F(SEQ ID NO: 21) | GFP2-PX12-20v7C-Stopt7(SEQ ID NO: 85) | pUCFa-GFP2 |
| GFP2-Nu11F(SEQ ID NO: 21) | GFP2-PX12-20C-Stopt7(SEQ ID NO: 86) | pUCFa-GFP2 |

TABLE 4
Combination of template plasmid and primer used in PCR amplification
of GFP2 gene for Yarrowia lipolytica expression

| Forward Primer | Reverse Primer | Template Plasmid |
|---|---|---|
| YH-GFP2-Nu11F (SEQ ID NO: 87) | YH-GFP2-R(SEQ ID NO: 94) | pUCFa-GFP2 |
| YH-GFP2-PX12-20NF (SEQ ID NO: 88) | YH-GFP2-R2(SEQ ID NO: 94) | pUCFa-GFP2 |
| YH-GFP2-ZX12-B15NF (SEQ ID NO: 89) | YH-GFP2-R2(SEQ ID NO: 94) | pUCFa-GFP2 |
| YH-GFP2-ZX12-B19NF (SEQ ID NO: 90) | YH-GFP2-R2(SEQ ID NO: 94) | pUCFa-GFP2 |
| YH-GFP2-ZX12-B27NF (SEQ ID NO: 91) | YH-GFP2-R2(SEQ ID NO: 94) | pUCFa GFP2 |
| YH-GFP2-ZX12-B33N(SEQ ID NO: 92) | YH-GFP2-R2(SEQ ID NO: 94) | pUCFa-GFP2 |
| YH-GFP2-ZX12-B35N(SEQ ID NO: 93) | YH-GFP2-R2(SEQ ID NO: 94) | pUCFa-GFP2 |

### (2) Expression of Each of Various Tagged Proteins by Cell-Free Expression System

PUREfrex 1.0 (GeneFrontier Corporation) was used as the cell-free expression system. Solution I attached to the Kit was molten at room temperature, and then left to stand on ice. Solution II and Solution III attached were molten on ice. Solution I, Solution II and Solution III were lightly vortexed, and then spun down by a desk centrifuge, thereafter 25 μl of sterile distilled water was added to each of Solution II and Solution III molten, and the resultant was vortexed and then spun down, and mixed with Solution I molten. This mixed solution was mixed well by vortexing. Predetermined amounts of the plasmid and sterile distilled water were split and taken in a sterile 1.5-μl Eppendorf tube, 8 μl of a mixed solution of Solutions I to III was further added, and the resultant was mixed by pipetting so that no bubbles occurred, and spun down by a desk centrifuge. Next, reaction was carried out in a water bath at 37° C. for 4 hours, to thereby express each protein. After completion of the reaction, 10 μl of sterile distilled water was added to the reaction product, thereafter 20 μl of 2×sample buffer (ATTO) was added and mixed, and then heated in a boiling bath for 10 minutes, to thereby provide a sample for SDS-PAGE.

### (3) Transformation of E. Coli for Protein Expression

A glycerol stock of E. coli BL21 (DE3) (Novagen) was inoculated in a sterile 14-ml polystyrene tube in which a 3-ml SOB medium (20 g/l Bacto tryptone, 5 g/l Bacto Yeast Extract, 10 mM NaCl, 2.5 mM KCl, 10 mM MgSO₄, 10 mM MgCl₂) was placed, and shake culture was carried out at 37° C. and 200 rpm overnight. After 0.2 ml of the pre-culture liquid was inoculated in a sterile Erlenmeyer flask in which a 100-ml SOB medium was placed, shake culture was carried out at 30° C. and 200 rpm. When the turbidity (OD 600) at a wavelength of 600 nm reached 0.4 to 0.6, culture was stopped by ice-cooling for 10 to 30 minutes. The culture liquid was transferred to a 50-ml conical tube, and centrifuged at 2,500×g and 4° C. for 10 minutes. The supernatant was discarded, 15 ml of TB (10 mM PIPES-KOH, pH 6.7, 15 mM CaCl₂), 0.25 M KCl, 55 mM MnCl₂) obtained by ice-cooling of a pellet was added, and the resulting mixture was mildly suspended. The suspension was centrifuged at 2,500×g and 4° C. for 10 minutes. The supernatant was discarded, 10 ml of TB ice-cooled was added to the pellet, and the resulting mixture was mildly suspended. To the mixture was added 700 μl of DMSO, and suspended with being ice-cooled. Competent cells were obtained by dispensing to a 1.5-ml microtube by 50 μl. The cells were frozen with liquid nitrogen, and then stored at −80° C. before use.

The resulting competent cells were molten on ice, 1 ng of the peptide-tagged protein expression plasmid for E. coli, produced above, was added thereto, and thereafter the resultant was mildly mixed and left to stand on ice for 30 minutes. The resulting mixture was treated (heat shock) at 42° C. for 45 seconds and then left to stand on ice for 5 minutes. After addition of 250 μl of SOC, the tube was horizontalized and shaken at 37° C. and 200 rpm for 1 hour. After 100 μl of the shaken product was applied to 2×YT agar medium containing 100 mg/l kanamycin, static culture was carried out at 37° C. overnight, to thereby obtain a transformed colony.

(4) Protein Induction Culture of E. coli

A single colony after transformation was smeared on a plate medium (2×YT, 100 mg/l kanamycin), and left to stand in an incubator at 37° C. overnight to perform culture. Next, bacterial cells were scraped with a sterile disposable loop from the plate medium after the culture, and inoculated into a sterile 14-ml polystyrene tube to which 2 ml of a pre-culture medium (2×YT, 100 mg/l kanamycin) was dispensed, and shake culture was performed at 37° C. and 200 rpm until the OD 600 value reached 0.6 to 1.0. The culture product was split and taken in a 1.5-ml Eppendorf tube in an amount so that the OD 600 value was 0.3 in addition of 1.0 ml of 2×YT medium (100 mg/l kanamycin) to a precipitated product obtained by removal of the centrifuged supernatant from the culture product, and then left to stand and held at 4° C. (in a refrigerator) overnight. On the next day, the sample was centrifuged at 2,000 rpm and 4° C. for 30 minutes and thereafter the supernatant was removed, and 1 ml of new 2×YT medium (100 mg/l kanamycin) was added thereto to suspend a precipitate. Furthermore, 300 μl of the 1 ml of the sample was inoculated to 2.7 ml of 2×YT medium (100 mg/l kanamycin) so that the OD 600 value was 0.03, and shake culture was carried out at 37° C. and 200 rpm until the OD 600 value reached 0.4 to 1.0. Next, 3 μl (final concentration 1 mM) of 1M IPTG (induction agent) was added, and shake culture was carried out at 30° C. and 200 rpm for 12 hours. After completion of the culture, a test tube where the sample was placed was cooled on ice for 5 minutes to stop amplification of E. coli, thereafter 200 μl of a culture liquid was split and taken in a new 1.5-ml Eppendorf tube, and centrifugation was carried out at 5,000 rpm and 4° C. for 5 minutes. Next, the supernatant was removed, and the bacterial cells was frozen by liquid nitrogen and then cryopreserved at −80° C.

(5) Extraction of Protein from E. coli

To the cryopreserved sample was added 100 μl of a sample buffer (EZ Apply, ATTO Corporation), and the resulting mixture was stirred in a vortex mixer and then heated in boiling water for 10 minutes to perform SDS treatment of the sample.

### (6) Western Analysis

Various protein purification preparations were each used for a standard substance in protein quantification. The preparation was repeatedly subjected to 2-fold dilution with 1×sample buffer (ATTO Corporation) to thereby produce a dilution series, and the dilution series was used for standards.

An electrophoresis tank (Criterion cell, BIO RAD) and Criterion TGX-gel (BIO RAD) were used for protein electrophoresis (SDS-PAGE). An electrophoresis buffer (Tris/Glycine/SDS Buffer, BIO RAD) was placed in the electrophoresis tank, 10 μl of the SDS-treated sample was applied to each well, and electrophoresis was carried out at a constant voltage of 200 V for 40 minutes.

The gel after the electrophoresis was subjected to blotting by Trans-Blot Turbo (BIO RAD) using a Trans-Blot Transfer Pack (BIO RAD).

The membrane after the blotting was immersed in a blocking solution (TBS system, pH 7.2, Nacalai Tesque, Inc), shaken at room temperature for 1 hour or left to stand at 4° C. for 16 hours, and then washed by shaking at room temperature in TBS-T (137 mM sodium chloride, 2.68 mM potassium chloride, 1% polyoxyethylene sorbitan monolaurate, 25 mM Tris-HCl, pH 7.4) for 5 minutes three times.

An antiserum Rabbit-monoclonal Anti-GFP antibody ab32146 (Abcam) for detection of a green fluorescent protein (GFP2), an antiserum Rabbit-monoclonal Anti-VHH antibody A01860 (GenScript) for detection of a VHH antibody (AmylD9), and an antiserum Rabbit-polyclonal Anti-XynA antibody (Scrum Inc.) for detection of xylanase (XynA) were each diluted 6,000-fold with TBS-T, and then used. The membrane was immersed in the dilution, shaken at room temperature for 2 hours to thereby allow antigen-antibody reaction to occur, and washed by shaking in TBS-T at room temperature for 5 minutes three times.

An Anti-Rabbit IgG, AP-linked Antibody #7054 (Cell Signaling), diluted 3,000-fold with TBS-T, was used for a secondary antibody. The membrane was immersed in the present dilution, shaken at room temperature for 1 hour to thereby allow antigen-antibody reaction to occur, and washed by shaking in TBS-T at room temperature for 5 minutes three times. Chromogenic reaction with alkaline phosphatase was carried out by immersing the membrane in a coloring solution (0.1 M sodium chloride, 5 mM magnesium chloride, 0.33 mg/ml nitroblue tetrazolium, 0.33 mg/ml 5-bromo-4-chloro-3-indolyl-phosphate, 0.1 M Tris-HCl, pH 9.5), and shaking the membrane at room temperature for 15 minutes, and the membrane was washed with distilled water, and then placed on KIMTOWEL and dried at room temperature.

An image of the membrane colored was taken at a resolution of 600 dpi with a scanner (PM-A900, Epson), and various proteins were each quantified with image analysis software (CS Analyzer ver. 3.0, ATTO Corporation).

### (7) Measurement of Fluorescence Intensity of GFP2 Protein

After 100 μl of an induced culture sample of GFP2 protein was split and taken in a 96-well microplate, and diluted 2-fold with sterile distilled water, the fluorescence intensity (λEm) at 510 nm was measured at an excitation wavelength (λEx) of 395 nm with a fluorescence microplate reader Spectra Max iD5 (Molecular DEVICES). Additionally, the OD value at 600 nm of the same sample was measured, and the amount of proliferation of E. coli was estimated. Next, the fluorescence intensity was divided by the OD value, and thus the fluorescence intensity per an OD value of 1.0 was calculated.

(8) Construction of E. coli-Yarrowia Lipolytica Shuttle Vector

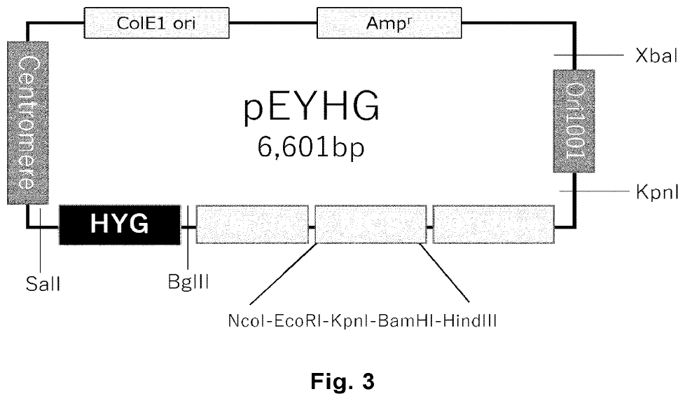

A plasmid composed of Ori-1001 (GenBank: EU340887.1) and Centromere 1.1 (GenBank: AF099207.1) according to plasmid replication in Yarrowia lipolytica, ColE1 on according to plasmid replication in E. coli, a hygromycin resistance gene (HYG), and a TEF promotor, a multicloning site and a CYC1 terminator according to metabolizing enzyme expression was synthesized by FASMAC, and thus plasmid 5 (pEYHG) was obtained (FIG. 3, SEQ ID NO:95).

### (9) Construction of Gene Expression Plasmids for Yarrowia Lipolytica, Encoding Various Tagged GFP2 Proteins

Plasmid 1 obtained by inserting an artificial synthetic DNA (SEQ ID NO:9) encoding a GFP2 protein, into the EcoRV recognition site of the pUC19-modified plasmid pUCFa (Fasmac), as in (1), was used as a template.

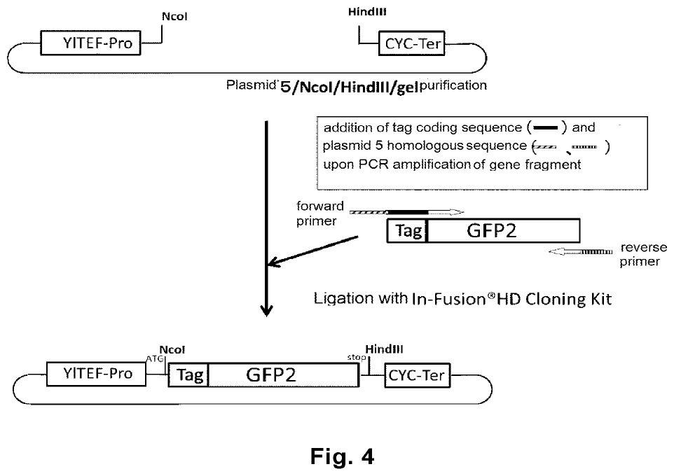

Specifically, PCR by the combination of a template plasmid DNA, a forward primer, and a reverse primer was performed, as shown in Table 4, for addition of each of various tags (Table 6) to the N-terminus of a GFP2 protein. A sequence homologous to plasmid 5 was added to the 5′-end of each primer. The resulting amplification fragment was purified by a QIAquick PCR Purification Kit (QIAGEN) and then inserted into plasmid 5 (pEYHG) digested with Not I and Hind III, by use of an In-Fusion HD Cloning Kit (TaKaRa), and thus a plasmid for expression was obtained (FIG. 4). Subsequently, the plasmid constructed was introduced to competent cells DH5-α (NIPPON GENE CO., LTD.), and cloning was performed. Next, the plasmid was extracted and the base sequence was confirmed, and thereafter the plasmid was used for transformation of yeast.

### (10) Transformation of Yarrowia Lipolytica

Yarrowia lipolytica was subjected to shake culture in 150 mL of a YPD-Rich medium (2% yeast extract, 4% peptone, 4% D-glucose, 0.01% Tryptophan, 0.002% Adenine) at 28° C. and 180 rpm for 16 to 18 hours. After the turbidity (OD 600) was confirmed to reach 16 to 24, the culture product was centrifuged, thereafter 1 M sorbitol was added to a precipitate for suspension, and centrifugation was again performed. After 1 M sorbitol was again added to the precipitate to suspend the bacterial cells, centrifugation was performed to remove the supernatant, thereafter not only 1 M sorbitol was added to, but also each of various plasmid DNA solutions constructed for transformation was added to the precipitate, and the resultant was mixed by a vortex mixer.

Two hundred μl of the suspension was dispensed to a 0.2-cm cuvette for electroporation (manufactured by Bio-Rad Laboratories, Gene Pulser Cuvette), and electroporation was carried out with Micro Pulser (manufactured by Bio-Rad Laboratories) at a voltage of 3.0 KV twice with respect to one sample. To 200 μl of the sample suspension was added 1,200 μl of the YPD-Rich medium, and shaken at 28° C. and 200 rpm for 1 hour. After the shaking, centrifugation was performed, 1 ml of 1 M sorbitol was added to a precipitate to suspend the precipitate, and the resultant was applied to a YPDm plate medium (0.2% yeast extract, 5% peptone, 0.1% D-glucose, 50 mM sodium-phosphate buffer pH 6.8, 2% Agar). Static culture was performed at 28° C. for 5 to 7 days to thereby obtain a transformed colony.

### (11) Culture and Sampling of Yarrowia Lipolytica

A clone where introduction of an objective gene could be confirmed by PCR was inoculated to a sterile 15-ml round tube where 4 mL of a 116YPD medium (1% peptone, 1% yeast extract, 6% glucose) was dispensed, in an amount so that the OD 600 was 0.1, and shake culture was performed at 28° C. and 200 rpm for a predetermined time.

After 2 days from the culture, 100 μl of the culture product was split and taken in a 1.5-mL Eppendorf tube, and adopted as a sample for western analysis of a GFP2 protein.

(12) Extraction of Enzyme from Yarrowia Lipolytica

A GFP2 protein was extracted by adding 1.0 mL of a 0.1N NaOH solution to the sample obtained in (11). According to a method of Akira Hosomi et al., (Akira Hosomi, et al: J Biol Chem, 285, (32), 24324-24334, 2010), suspending the bacterial cells by a vortex mixer and then leaving them to stand under ice-cooling for 10 minutes. Next, centrifugation was performed at 4° C. and 15,000 g for 5 minutes and the supernatant was discarded, and thereafter the resulting precipitate was recovered.

### (13) Western Analysis

To the resulting precipitate of the GFP2 protein was added 100 μl of a sample buffer (EZ Apply, manufactured by ATTO Corporation), and the resulting mixture was stirred in a vortex mixer and then warmed in boiling water for 10 minutes to perform SDS treatment of the sample. Subsequently, electrophoresis (SDS-PAGE) and blotting were performed by the same method as in (6), with purified GFP as a preparation.

Also after the blotting, the membrane was immersed in a blocking solution (TBS system, pH 7.2, Nacalai Tesque, Inc) and shaken at room temperature for 1 hour, and then washed by shaking in TBS-T (137 mM sodium chloride, 2.68 mM potassium chloride, 1% polyoxyethylene sorbitan monolaurate, 25 mM Tris-HCl, pH 7.4) at room temperature for 5 minutes three times, in the same manner as in (6). An antiserum Rabbit-monoclonal Anti-GFP antibody ab32146 (Abcam) diluted 3,000-fold with TBS-T was used for detection of the GFP2 protein. The membrane was immersed in the present dilution, shaken at room temperature for 2 hours to thereby allow antigen-antibody reaction to occur, and washed by shaking in TBS-T at room temperature for 5 minutes three times. An Anti-Rabbit IgG, AP-linked Antibody #7054 (Cell Signaling), was used for a secondary antibody. An image of the membrane colored was taken at a resolution of 600 dpi with a scanner (PM-A900, Epson), and the expression level of each of various enzymes was measured with image analysis software (CS Analyzer ver. 3.0, ATTO Corporation).

### (14) Results

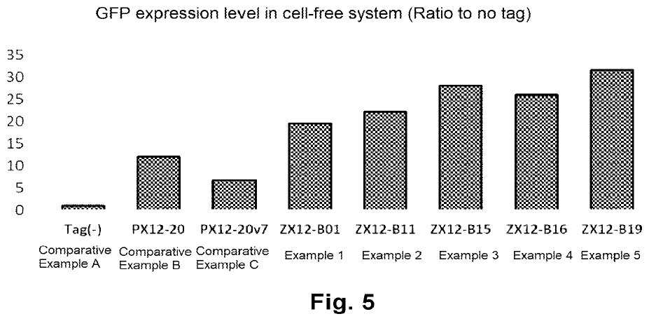

The results are shown in FIGS. 5 to 14.

As illustrated in FIG. 5, the expression level of the fusion protein where the peptide tag with G, E or Q arranged at certain intervals, of each of Examples 1 to 4, was linked to the N-terminus of GFP, in the cell-free expression system, was extremely improved as compared with that of the fusion protein where the peptide tag with P arranged at certain intervals, of each of Comparative Examples, was linked to the N-terminus of GFP.

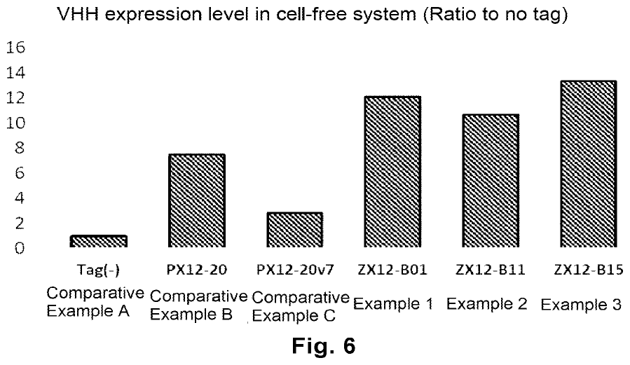

As illustrated in FIG. 6, the expression level of the fusion protein where the peptide tag with G, E or Q arranged at certain intervals, of each of Examples 1 to 3, was linked to the N-terminus of the VHH antibody, in the cell-free expression system, was extremely improved as compared with that of the fusion protein where the peptide tag with P arranged at certain intervals, of each of Comparative Examples, was linked to the N-terminus of the VHH antibody.

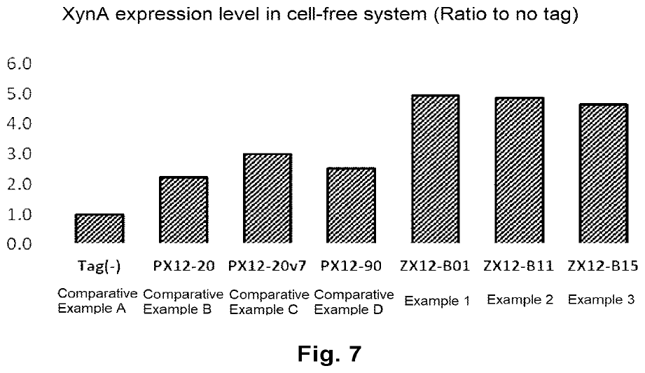

As illustrated in FIG. 7, the expression level of the fusion protein where the peptide tag with G, E or Q arranged at certain intervals, of each of Examples 1 to 3, was linked to the N-terminus of XynA, in the cell-free expression system, was extremely improved as compared with that of the fusion protein where the peptide tag with P arranged at certain intervals, of each of Comparative Examples, was linked to the N-terminus of XynA.

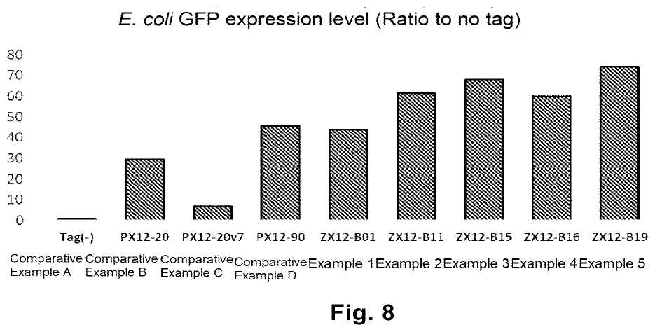

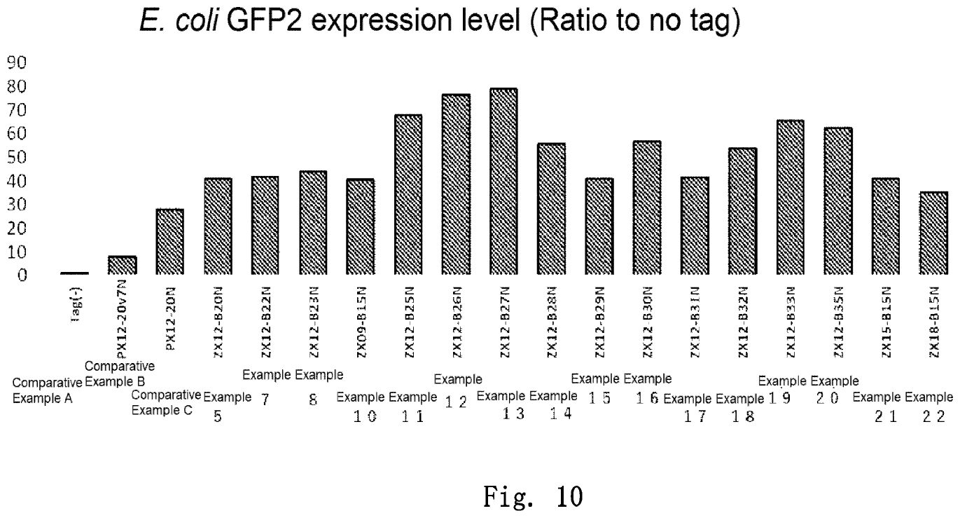

As illustrated in FIGS. 8 and 10, the expression level of the fusion protein where the peptide tag with G, E or Q arranged at certain intervals, of each of Examples 1 to 22, was linked to the N-terminus of GFP, in the E. coli expression system, was extremely improved as compared with that of the fusion protein where the peptide tag with P arranged at certain intervals, of each of Comparative Examples, was linked to the N-terminus of GFP.

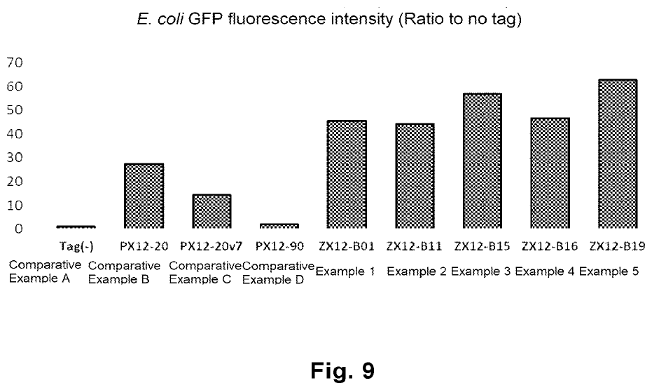

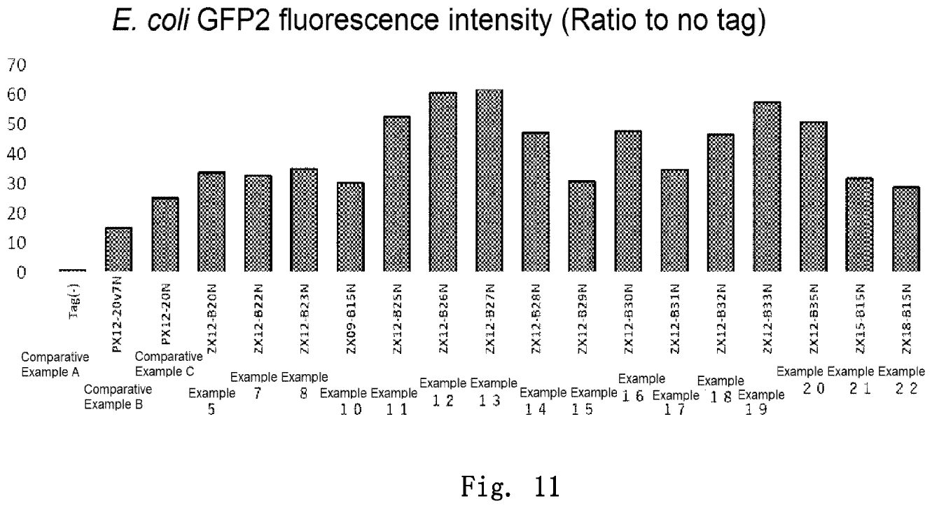

As illustrated in FIGS. 9 and 11, it could be confirmed that the fluorescence intensity of GFP exhibited an extremely high value in a fusion protein where the peptide tag with G, E or Q arranged at certain intervals, of each of Examples 1 to 22, was linked to the N-terminus of GFP, and a functional protein was expressed.

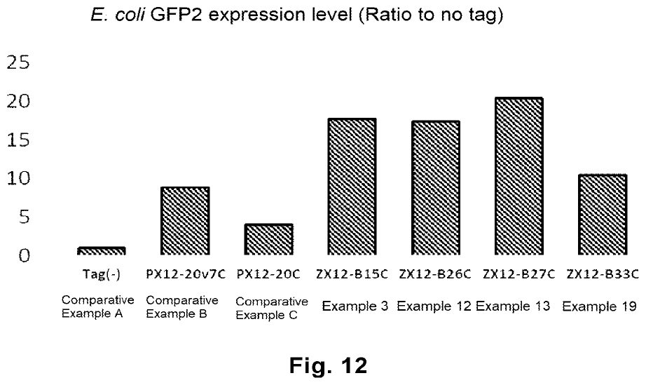

As illustrated in FIG. 12, the expression level of the fusion protein where the peptide tag with G, E or Q arranged at certain intervals, of each of Examples 3, 12, 13, and 19, was linked to the C-terminus of GFP, in the E. coli expression system, was extremely improved as compared with that of the fusion protein where the peptide tag with P arranged at certain intervals, of each of Comparative Examples, was linked to the C-terminus of GFP.

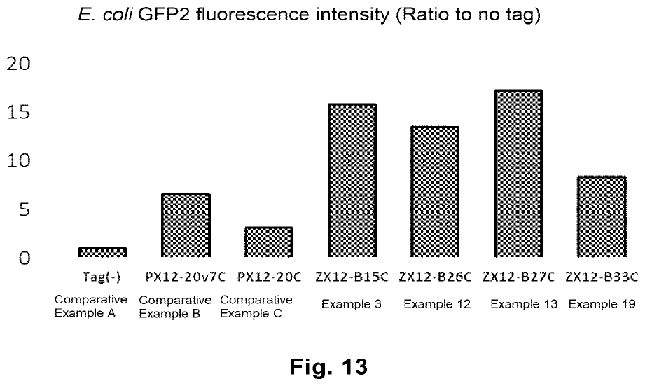

As illustrated in FIG. 13, it could be confirmed that the fluorescence intensity of GFP exhibited an extremely high value in a fusion protein where the peptide tag with G, E or Q arranged at certain intervals, of each of Examples 3, 12, 13, and 19, was linked to the C-terminus of GFP, and a functional protein was expressed.

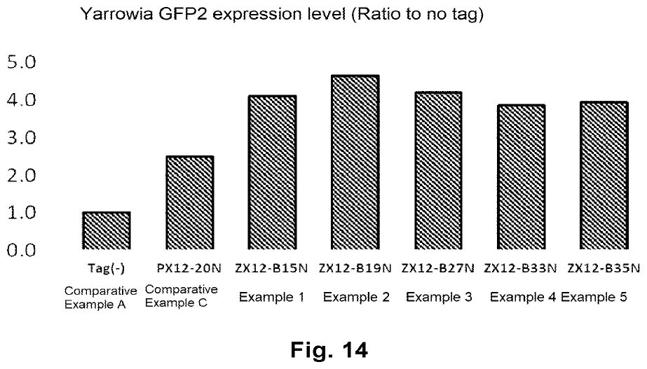

As illustrated in FIG. 14, the expression level of the fusion protein where the peptide tag with G, E or Q arranged at certain intervals, of each of Examples 3, 4, 13, 19 and 20, was linked to the N-terminus of GFP, in the Yarrowia lipolytica expression system, was extremely improved as compared with that of the fusion protein where the peptide tag with P arranged at certain intervals, of each of Comparative Examples, was linked to the N-terminus of GFP.

### INDUSTRIAL APPLICABILITY

The peptide tag of the present invention is useful in the fields of genetic engineering, protein engineering, and the like, and a protein to which the peptide tag of the present invention is added is useful in the fields of medical treatment, research, food product, farming, and the like.

1. A peptide of 6 to 50 amino acid residues comprising the following sequence:
Xₘ(JYₙ)_{q}JZᵣ (I)
wherein J is an amino acid residue selected from Q (glutamine), E (glutamic acid), and G (glycine);
X and Y are each an amino acid residue independently selected from arginine (R), glycine (G), serine (S), lysine (K), threonine (T), leucine (L), asparagine (N), glutamine(Q), histidine (H), proline (P), isoleucine (I), valine (V), alanine (A), and methionine (M) with the proviso that X and Y are each other than Q in the case of said peptide containing Q as J and X and Y are each other than G in the case of said peptide containing G as J, and
at least one Y in each repeating unit JYₙ is K, L, N, Q, H or R;
Z is an amino acid residue independently selected from R, G, S, K, T, N, Q, H and P with the proviso that Z is other than Q in the case of said peptide containing Q as J and Z is other than G in the case of said peptide containing G as J;
the number of P's contained in the peptide is 0 or 1; and
m is an integer of 0 to 6, n is 1, 2 or 3, q is an integer of 1 to 10, and r is an integer of 0 to 10.

2. The peptide according to claim 1, comprising the sequence selected from the following (1) to (3):
(1) Xₘ(QYₙ)_{q}QZᵣ
(2) Xₘ(EYₙ)_{q}EZᵣ
(3) Xₘ(GYₙ)_{q}GZᵣ
in (1), X and Y are each an amino acid residue independently selected from R, G, S, K, T, L, N, H and P and at least one Y contains K, L, N, H or R, and Z is an amino acid residue independently selected from R, G, S, K, T, N, H and P;
in (2), X and Y are each an amino acid residue independently selected from R, G, S, K, T, L, N, Q, H and P and at least one Y contains K, L, N, Q, H or R, and Z is an amino acid residue independently selected from R, G, S, K, T, N, Q, H and P; and
in (3), X and Y are each an amino acid residue independently selected from R, S, K, T, L, N, Q, H and P and at least one Y contains K, L, N, Q, H or R, and Z is an amino acid residue independently selected from R, S, K, T, N, Q, H and P.

3. The peptide according to claim 2, wherein
in (1), X and Y are each an amino acid residue independently selected from R, K and N and at least one Y contains R, K or N,
in (2), X and Y are each an amino acid residue independently selected from R, K, N and Q and at least one Y contains R, K, N or Q, and
in (3), X and Y are each an amino acid residue independently selected from R, K, N and Q and at least one Y contains R, K, N or Q.

4. The peptide according to claim 3, wherein
in (1), Xₘ is (R/G/S/I/V/T/N/H/P/A/M)(K/N)(K/N), Yₙ is (K/N)(K/N), and Zᵣ is RS, NKPRS (SEQ ID NO:45) or KNPRS (SEQ ID NO:46),
in (2), Xₘ, is ((R/G/S/I/V/T/N/H/P/A/M)(K/N/Q)(K/N), Yₙ is (K/N/Q)(K/N), and Zᵣ is RS, KNPRS (SEQ ID NO:46) or QNPRS (SEQ ID NO:64), and
in (3), Xₘ is (R/S/1/V/T/N/H/P/A/M)(K/N/Q)(K/N), Yₙ is (K/N/Q)(K/N), and Zᵣ is RS, NKPRS (SEQ ID NO:45) or KNPRS (SEQ ID NO:46).

5. The peptide according to claim 1, wherein n is 2 or 3.

6. The peptide according to claim 1, wherein q is an integer of 2 to 5.

7. The peptide according to claim 1, comprising the amino acid sequence selected from SEQ ID NOs:1 to 4 and SEQ ID NOs:47 to 62.

8. A tagged protein comprising the peptide according to claim 1 and a useful protein.

9. The tagged protein according to claim 8, wherein the useful protein is an enzyme, a cytokine, an antibody, or a fluorescent protein.

10. A DNA encoding the tagged protein according to claim 8.

11. A recombinant vector comprising the DNA according to claim 10.

12. A transformant transformed with the DNA according to claim 10.

13. A method of producing a tagged protein, comprising culturing the transformant according to claim 12 and expressing and accumulating a tagged protein, and recovering the tagged protein.

14. A method of producing a tagged protein, comprising introducing the DNA according to claim 10 or an RNA transferred therefrom into a cell-free expression system and expressing and accumulating a tagged protein, and recovering the tagged protein.

15. A transformant transformed with the recombinant vector according to claim 11.


SEQUENCE LISTING (95 sequences)

SEQ ID NO: 1 - protein, 12 aa, Artificial Sequence
  ZX12-B01
RKNGKNGKNGRS

SEQ ID NO: 2 - protein, 12 aa, Artificial Sequence
  ZX12-B11
RKNEKNEKNERS

SEQ ID NO: 3 - protein, 12 aa, Artificial Sequence
  ZX12-B15
RNKQNKQNKQRS

SEQ ID NO: 4 - protein, 12 aa, Artificial Sequence
  ZX12-B19
RQNEQNEQNERS

SEQ ID NO: 5 - DNA, 36 bp, Artificial Sequence
  ZX12-B01
cgtaaaaatggtaaaaatggtaaaaatggtcgttca

SEQ ID NO: 6 - DNA, 36 bp, Artificial Sequence
  ZX12-B11
cgtaaaaatgaaaaaaatgaaaaaaatgaacgttca

SEQ ID NO: 7 - DNA, 36 bp, Artificial Sequence
  ZX12-B15
cgtaataaacaaaataaacaaaataaacaacgttca

SEQ ID NO: 8 - DNA, 36 bp, Artificial Sequence
  ZX12-B19
cgtcaaaatgaacaaaatgaacaaaatgaacgttca

SEQ ID NO: 9 - DNA, 717 bp, Artificial Sequence
  GFP2
  CDS (1)..(714)
atgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtga

SEQ ID NO: 10 - protein, 238 aa, Artificial Sequence
  Synthetic Construct
MSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYR

SEQ ID NO: 11 - DNA, 561 bp, Bacillus subtilis
  CDS (1)..(558)
atggctagcccagactactggcaaaattggactgatggcggcggaacagtaaacgctgtcaatgggcctggagggaattacagtgttaattggtctaataccggaaatttcgttgttggtaaaggttggactacaggttcgccatctaggacaataaactataatgccggagtttgggcgccgaatggcaatggatatttggctttatatggttggacgagatcacctctcatagaatattatgtagtggattcatggggtacttatagacctactggaacgtataaaggtactgtaaaaagtgatggcggcacatatgacatatatacaactacacgttataatgcaccttccattgatggcgaaaaaactactttcacgcagtactggagtgttcgccagacgaagagaccaactggaagcaacgctaaaatcactttcagcaatcatgttagagcatggaagagtcatggaatgaatctgggtagtatttggtcttatcaagtcttagcgacagagggatatcaaagtagtggaagttctaacgtaacagtgtggtga

SEQ ID NO: 12 - protein, 186 aa, Bacillus subtilis
MASPDYWQNWTDGGGTVNAVNGPGGNYSVNWSNTGNFVVGKGWTTGSPSRTINYNAGVWAPNGNGYLALYGWTRSPLIEYYVVDSWGTYRPTGTYKGTVKSDGGTYDIYTTTRYNAPSIDGEKTTFTQYWSVRQTKRPTGSNAKITFSNHVRAWKSHGMNLGSIWSYQVLATEGYQSSGSSNVTVW

SEQ ID NO: 13 - DNA, 366 bp, Artificial Sequence
  VHH
  CDS (1)..(363)
atgcaggttcagttggtcgaaagtggaggtggaagcgttcaagctggtggctccttatccctgtcatgtgcggcttctacctatacggataccgtaggatggtttcgtcaagcgcctggtaaggaacgcgaaggtgttgcggcgatttatcgccgtacaggctacacgtatagtgccgacagcgtcaaagggcgctttaccctctctcaggacaacaacaagaacaccgtgtatctgcagatgaactcgctcaaaccggaagatactggcatctactactgcgccactggcaatagcgtgcgtttagcgtcttgggaaggctacttctattggggtcaaggcacacaggttacggtgtcgtcttga

SEQ ID NO: 14 - protein, 121 aa, Artificial Sequence
  Synthetic Construct
MQVQLVESGGGSVQAGGSLSLSCAASTYTDTVGWFRQAPGKEREGVAAIYRRTGYTYSADSVKGRFTLSQDNNKNTVYLQMNSLKPEDTGIYYCATGNSVRLASWEGYFYWGQGTQVTVSS

SEQ ID NO: 15 - protein, 12 aa, Artificial Sequence
  PX12-20
RKPGKGPGKPRS

SEQ ID NO: 16 - protein, 12 aa, Artificial Sequence
  PX12-20v7
RKPKKKPKKPRS

SEQ ID NO: 17 - protein, 12 aa, Artificial Sequence
  PX12-90
RQPQQQPQQPRS

SEQ ID NO: 18 - DNA, 36 bp, Artificial Sequence
  PX12-20
cgtaaaccaggtaaaggtccaggtaaaccacgttca

SEQ ID NO: 19 - DNA, 36 bp, Artificial Sequence
  PX12-20v7
cgtaaaccaaaaaaaaaaccaaaaaaaccacgttca

SEQ ID NO: 20 - DNA, 36 bp, Artificial Sequence
  PX12-90
cgtcaaccacaacaacaaccacaacaaccacgttca

SEQ ID NO: 21 - DNA, 40 bp, Artificial Sequence
  GFP2-NullF
aagaaggagatataccatgagcgggggcgaggagctgttc

SEQ ID NO: 22 - DNA, 76 bp, Artificial Sequence
  GFP2-ZX12-B01NF
aagaaggagatataccatgcgtaaaaatggtaaaaatggtaaaaatggtcgttcaagcgggggcgaggagctgttc

SEQ ID NO: 23 - DNA, 76 bp, Artificial Sequence
  GFP2-ZX12-B11NF
aagaaggagatataccatgcgtaaaaatgaaaaaaatgaaaaaaatgaacgttcaagcgggggcgaggagctgttc

SEQ ID NO: 24 - DNA, 76 bp, Artificial Sequence
  GFP2-ZX12-B15NF
aagaaggagatataccatgcgtaataaacaaaataaacaaaataaacaacgttcaagcgggggcgaggagctgttc

SEQ ID NO: 25 - DNA, 76 bp, Artificial Sequence
  GFP2-ZX12-B19NF
aagaaggagatataccatgcgtcaaaatgaacaaaatgaacaaaatgaacgttcaagcgggggcgaggagctgttc

SEQ ID NO: 26 - DNA, 76 bp, Artificial Sequence
  GFP2-PX12-20NF
aagaaggagatataccatgcgtaaaccaggtaaaggtccaggtaaaccacgttcaagcgggggcgaggagctgttc

SEQ ID NO: 27 - DNA, 76 bp, Artificial Sequence
  GFP2-PX12-20v7NF
aagaaggagatataccatgcgtaaaccaaaaaaaaaaccaaaaaaaccacgttcaagcgggggcgaggagctgttc

SEQ ID NO: 28 - DNA, 76 bp, Artificial Sequence
  GFP2-PX12-90NF
aagaaggagatataccatgcgtcaaccacaacaacaaccacaacaaccacgttcaagcgggggcgaggagctgttc

SEQ ID NO: 29 - DNA, 43 bp, Artificial Sequence
  GFP2-stopT7
tcgagtgcggccgcaagctttcacctgtacagctcgtccatgc

SEQ ID NO: 30 - DNA, 39 bp, Artificial Sequence
  VHH-NullF
aagaaggagatataccatgcaggttcagttggtcgaaag

SEQ ID NO: 31 - DNA, 75 bp, Artificial Sequence
  VHH-ZX12-01NF
aagaaggagatataccatgcgtaaaaatggtaaaaatggtaaaaatggtcgttcacaggttcagttggtcgaaag

SEQ ID NO: 32 - DNA, 75 bp, Artificial Sequence
  VHH-ZX12-11NF
aagaaggagatataccatgcgtaaaaatgaaaaaaatgaaaaaaatgaacgttcacaggttcagttggtcgaaag

SEQ ID NO: 33 - DNA, 75 bp, Artificial Sequence
  VHH-ZX12-15NF
aagaaggagatataccatgcgtaataaacaaaataaacaaaataaacaacgttcacaggttcagttggtcgaaag

SEQ ID NO: 34 - DNA, 75 bp, Artificial Sequence
  VHH-PX12-20NF
aagaaggagatataccatgcgtaaaccaggtaaaggtccaggtaaaccacgttcacaggttcagttggtcgaaag

SEQ ID NO: 35 - DNA, 75 bp, Artificial Sequence
  VHH-PX12-20v7NF
aagaaggagatataccatgcgtaaaccaaaaaaaaaaccaaaaaaaccacgttcacaggttcagttggtcgaaag

SEQ ID NO: 36 - DNA, 43 bp, Artificial Sequence
  AmylD9-stopT7
tcgagtgcggccgcaagctttcaagacgacaccgtaacctgtg

SEQ ID NO: 37 - DNA, 38 bp, Artificial Sequence
  XynA-NullF
aagaaggagatataccatggctagcccagactactggc

SEQ ID NO: 38 - DNA, 74 bp, Artificial Sequence
  XynA-ZX12-01NF
aagaaggagatataccatgcgtaaaaatggtaaaaatggtaaaaatggtcgttcagctagcccagactactggc

SEQ ID NO: 39 - DNA, 74 bp, Artificial Sequence
  XynA-ZX12-11NF
aagaaggagatataccatgcgtaaaaatgaaaaaaatgaaaaaaatgaacgttcagctagcccagactactggc

SEQ ID NO: 40 - DNA, 74 bp, Artificial Sequence
  XynA-ZX12-15NF
aagaaggagatataccatgcgtaataaacaaaataaacaaaataaacaacgttcagctagcccagactactggc

SEQ ID NO: 41 - DNA, 74 bp, Artificial Sequence
  XynA-PX12-20NF
aagaaggagatataccatgcgtaaaccaggtaaaggtccaggtaaaccacgttcagctagcccagactactggc

SEQ ID NO: 42 - DNA, 74 bp, Artificial Sequence
  XynA-PX12-20v7NF
aagaaggagatataccatgcgtaaaccaaaaaaaaaaccaaaaaaaccacgttcagctagcccagactactggc

SEQ ID NO: 43 - DNA, 74 bp, Artificial Sequence
  XynA-PX12-90NF
aagaaggagatataccatgcgtcaaccacaacaacaaccacaacaaccacgttcagctagcccagactactggc

SEQ ID NO: 44 - DNA, 42 bp, Artificial Sequence
  XynA-stopT7
tcgagtgcggccgcaagctttcaccacactgttacgttagaa

SEQ ID NO: 45 - protein, 5 aa, Artificial Sequence
  partial sequence
NKPRS

SEQ ID NO: 46 - protein, 5 aa, Artificial Sequence
  partial sequence
KNPRS

SEQ ID NO: 47 - protein, 12 aa, Artificial Sequence
  ZX12-B20
RNKPNKQNKQRS

SEQ ID NO: 48 - protein, 12 aa, Artificial Sequence
  ZX12-B22
RNKQNKQNKPRS

SEQ ID NO: 49 - protein, 12 aa, Artificial Sequence
  ZX12-B23
RQNPQNEQNERS

SEQ ID NO: 50 - protein, 9 aa, Artificial Sequence
  ZX09-B15
RNKQNKQRS

SEQ ID NO: 51 - protein, 12 aa, Artificial Sequence
  ZX12-B25
GNKQNKQNKQRS

SEQ ID NO: 52 - protein, 12 aa, Artificial Sequence
  ZX12-B26
SNKQNKQNKQRS

SEQ ID NO: 53 - protein, 12 aa, Artificial Sequence
  ZX12-B27
INKQNKQNKQRS

SEQ ID NO: 54 - protein, 12 aa, Artificial Sequence
  ZX12-B28
VNKQNKQNKQRS

SEQ ID NO: 55 - protein, 12 aa, Artificial Sequence
  ZX12-B29
TNKQNKQNKQRS

SEQ ID NO: 56 - protein, 12 aa, Artificial Sequence
  ZX12-B30
NNKQNKQNKQRS

SEQ ID NO: 57 - protein, 12 aa, Artificial Sequence
  ZX12-B31
HNKQNKQNKQRS

SEQ ID NO: 58 - protein, 12 aa, Artificial Sequence
  ZX12-B32
PNKQNKQNKQRS

SEQ ID NO: 59 - protein, 12 aa, Artificial Sequence
  ZX12-B33
ANKQNKQNKQRS

SEQ ID NO: 60 - protein, 12 aa, Artificial Sequence
  ZX12-B35
MNKQNKQNKQRS

SEQ ID NO: 61 - protein, 15 aa, Artificial Sequence
  ZX15-B15
RNKQNKQNKQNKQRS

SEQ ID NO: 62 - protein, 18 aa, Artificial Sequence
  ZX18-B15
RNKQNKQNKQNKQNKQRS

SEQ ID NO: 63 - protein, 6 aa, Artificial Sequence
  partial sequence
RQNPQN

SEQ ID NO: 64 - protein, 5 aa, Artificial Sequence
  partial sequence
QNPRS

SEQ ID NO: 65 - DNA, 76 bp, Artificial Sequence
  GFP2-ZX12-B20NF
aagaaggagatataccatgcgtaataaaccaaataaacaaaataaacaacgttcaagcgggggcgaggagctgttc

SEQ ID NO: 66 - DNA, 76 bp, Artificial Sequence
  GFP2-ZX12-B22NF
aagaaggagatataccatgcgtaataaacaaaataaacaaaataaaccacgttcaagcgggggcgaggagctgttc

SEQ ID NO: 67 - DNA, 76 bp, Artificial Sequence
  GFP2-ZX12-B23NF
aagaaggagatataccatgcgtcaaaatccacaaaatgaacaaaatgaacgttcaagcgggggcgaggagctgttc

SEQ ID NO: 68 - DNA, 67 bp, Artificial Sequence
  GFP2-ZX09-B15NF
aagaaggagatataccatgcgtaataaacaaaataaacaacgttcaagcgggggcgaggagctgttc

SEQ ID NO: 69 - DNA, 76 bp, Artificial Sequence
  GFP2-ZX12-B25NF
aagaaggagatataccatgggtaataaacaaaataaacaaaataaacaacgttcaagcgggggcgaggagctgttc

SEQ ID NO: 70 - DNA, 76 bp, Artificial Sequence
  GFP2-ZX12-B26NF
aagaaggagatataccatgtctaataaacaaaataaacaaaataaacaacgttcaagcgggggcgaggagctgttc

SEQ ID NO: 71 - DNA, 76 bp, Artificial Sequence
  GFP2-ZX12-B27NF
aagaaggagatataccatgataaataaacaaaataaacaaaataaacaacgttcaagcgggggcgaggagctgttc

SEQ ID NO: 72 - DNA, 76 bp, Artificial Sequence
  GFP2-ZX12-B28NF
aagaaggagatataccatggtaaataaacaaaataaacaaaataaacaacgttcaagcgggggcgaggagctgttc

SEQ ID NO: 73 - DNA, 76 bp, Artificial Sequence
  GFP2-ZX12-B29NF
aagaaggagatataccatgactaataaacaaaataaacaaaataaacaacgttcaagcgggggcgaggagctgttc

SEQ ID NO: 74 - DNA, 76 bp, Artificial Sequence
  GFP2-ZX12-B30NF
aagaaggagatataccatgaataataaacaaaataaacaaaataaacaacgttcaagcgggggcgaggagctgttc

SEQ ID NO: 75 - DNA, 76 bp, Artificial Sequence
  GFP2-ZX12-B31NF
aagaaggagatataccatgcataataaacaaaataaacaaaataaacaacgttcaagcgggggcgaggagctgttc

SEQ ID NO: 76 - DNA, 76 bp, Artificial Sequence
  GFP2-ZX12-B32NF
aagaaggagatataccatgcctaataaacaaaataaacaaaataaacaacgttcaagcgggggcgaggagctgttc

SEQ ID NO: 77 - DNA, 76 bp, Artificial Sequence
  GFP2-ZX12-B33NF
aagaaggagatataccatggctaataaacaaaataaacaaaataaacaacgttcaagcgggggcgaggagctgttc

SEQ ID NO: 78 - DNA, 76 bp, Artificial Sequence
  GFP2-ZX12-B35NF
aagaaggagatataccatgatgaataaacaaaataaacaaaataaacaacgttcaagcgggggcgaggagctgttc

SEQ ID NO: 79 - DNA, 85 bp, Artificial Sequence
  GFP2-ZX15-B15NF
aagaaggagatataccatgcgtaataaacaaaataaacaaaataaacaaaataaacaacgttcaagcgggggcgaggagctgttc

SEQ ID NO: 80 - DNA, 94 bp, Artificial Sequence
  GFP2-ZX18-B15NF
aagaaggagatataccatgcgtaataaacaaaataaacaaaataaacaaaataaacaaaataaacaacgttcaagcgggggcgaggagctgttc

SEQ ID NO: 81 - DNA, 79 bp, Artificial Sequence
  GFP2-ZX12-B15C-Stopt7
tcgagtgcggccgcaagctttcatgaacgttgtttattttgtttattttgtttattacgcctgtacagctcgtccatgc

SEQ ID NO: 82 - DNA, 79 bp, Artificial Sequence
  GFP2-ZX12-B26C-Stopt7
tcgagtgcggccgcaagctttcatgaacgttgtttattttgtttattttgtttattagacctgtacagctcgtccatgc

SEQ ID NO: 83 - DNA, 79 bp, Artificial Sequence
  GFP2-ZX12-B27C-Stopt7
tcgagtgcggccgcaagctttcatgaacgttgtttattttgtttattttgtttatttatcctgtacagctcgtccatgc

SEQ ID NO: 84 - DNA, 79 bp, Artificial Sequence
  GFP2-ZX12-B33C-Stopt7
tcgagtgcggccgcaagctttcatgaacgttgtttattttgtttattttgtttattagccctgtacagctcgtccatgc

SEQ ID NO: 85 - DNA, 79 bp, Artificial Sequence
  GFP2-PX12-20v7C-Stopt7
tcgagtgcggccgcaagctttcatgaacgtggtttttttggttttttttttggtttacgcctgtacagctcgtccatgc

SEQ ID NO: 86 - DNA, 79 bp, Artificial Sequence
  GFP2-PX12-20C-Stopt7
tcgagtgcggccgcaagctttcatgaacgtggtttacctggacctttacctggtttacgcctgtacagctcgtccatgc

SEQ ID NO: 87 - DNA, 36 bp, Artificial Sequence
  YH-GFP2-NullF
atcattcaaaccatgagcgggggcgaggagctgttc

SEQ ID NO: 88 - DNA, 72 bp, Artificial Sequence
  YH-GFP2-PX12-20NF
atcattcaaaccatgcgaaaaccaggtaaaggtccaggtaaaccaagatctagcgggggcgaggagctgttc

SEQ ID NO: 89 - DNA, 72 bp, Artificial Sequence
  YH-GFP2-ZX12-B15NF
atcattcaaaccatgcgaaataaacaaaataaacaaaataaacaaagatctagcgggggcgaggagctgttc

SEQ ID NO: 90 - DNA, 72 bp, Artificial Sequence
  YH-GFP2-ZX12-B19NF
atcattcaaaccatgcgacaaaatgaacaaaatgaacaaaatgaaagatctagcgggggcgaggagctgttc

SEQ ID NO: 91 - DNA, 72 bp, Artificial Sequence
  YH-GFP2-ZX12-B27NF
atcattcaaaccatgataaataaaccaaataaacaaaataaacaaagatctagcgggggcgaggagctgttc

SEQ ID NO: 92 - DNA, 72 bp, Artificial Sequence
  YH-GFP2-ZX12-B33NF
atcattcaaaccatggctaataaacaaaataaacaaaataaacaaagatctagcgggggcgaggagctgttc

SEQ ID NO: 93 - DNA, 72 bp, Artificial Sequence
  YH-GFP2-ZX12-B35NF
atcattcaaaccatgatgaataaacaaaataaacaaaataaacaaagatctagcgggggcgaggagctgttc

SEQ ID NO: 94 - DNA, 41 bp, Artificial Sequence
  YH-GFP2-R2
ttgtagacgcaagctttcacctgtacagctcgtccatgccg

SEQ ID NO: 95 - DNA, 6601 bp, Artificial Sequence
  pEYHG
tgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagcctaggtcgaagcaacgcgtggagagtttgggttttgggttacgtacgtagagccgtttgatagatggtacatccaccggctagcggaacacagtgtcaagacaagcctgcaacacagtcataatatttgcgatattcaggcgtatcaggtacaatctgaggtgtctcacaagtgccgtgcagtcccgcccccacttgcttctctttgtgtgtagtgtacgtacattatcgagaccgttgttcccgcccacctcgatccggggtcctatgcatccctgaaacattgattggaaattaacatatgagctgcgtgctttttgcattcaagggcgcagcttatcttgtatccttaattacacatgacctcttgagcgccacggtacattcctggcgtcagttcggtggagcggacacttttctctcctttgtctgacatgttggttaagttgtagtccagggacacaaggggttccaacggcagtggcagcctaccccacgctacccaccactggccctggtctaacttcgacgatcggcatcagggttcatggataggcggtgtgatttacgatgtgatggacaatgttagagagatcccactacttgtagtcaggccatcttttacgtacgcactgtaccatgatgtcaatggagtatgatgaaccgactttgagagactcacatctgcacaacaccatgtttcagcggaatccgacttccaacccaaacccaagcccctgtcagatatcgtgagaaggcacggcaccaactaatgcacacactccacctgtattgcaccaagataatgagggcatcgtcttggcgcgtcttggcgagagccgtgtttcgtgacgcaatcagagcagtttctggatagtatcttgtccagaaacacgatataaaccccatcgacgggcccgttgaagagcaccaacccactatccaatcctccaatccaacaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgagcaattaacagatagtttgccggtgataattctcttaacctcccacactcctttgacataacgatttatgtaacgaaactgaaatttgaccagatattgttgtaaatagaaaatctggcttgtaggtggcaaaatcccgtctttgttcatcaattccctctgtgactactcgtcatccctttatgttcgactgtcgtatttttattttccatacatacgcaagtgagatgcccgtgtccgaattcggcagtcgcatggactatcggttgctctgtacatacacatacgcgaggctggaacctatatcagagcactacttgtagggttgatgtaacattcaagaaaagcgcaagccagtgggtgatgtatagcagctacagcaactactgctcaacatgaaaaaggagggtgttaagacggccaagactgctttcttgctacgcctgagcaacgtagctctgcaacagagcaacagataatcgcctacggagacagagacagatctagagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaccatgggatccgctagcaagcttgcgtctacaactggacccttagcctgtatatatcaattgattatttaaagatttggtcggtaggcggttcgtattgtacaatgggatctgttactgaggtggatctacccaacttgcgagattcaattgcgagattcaatcgcgagattcaattgcgagaatcagttgcgagttgttctaacactcagcttctacgagcgcttgtattaggacgagtgatactccgtggggcgacggcttctcttgcgtcttctgttgtattctttcttacactatcgtccatctccaaccacctcgtacgaattcggagaaaactcaccgaggcagttccggtaccgagacaataacggaggatatgtctgataaaaggatgtaacataggcaagctgctcgtgagtgttgagtacgaaccttagatccaaatcacccgcacccacggatatacttgcttgaatatacagtagtatgtctagaatcagatcggaagagcgtcgttaagggaaagagtgttccgtactcagagagctattacaattcactggccgtcgttttacaacgtcgtgactgggaaaacccaggcgttacccaacttaatcgccttgcagcacatcctccgtttgcgagttggcggaatagcgaagaagcgcgtacagatcgtccgtcacagcagttgcgctctctgtaacgtaccgcagttcaaagtctacacctacaaacgcgaaagtcgctatcgcctgtttgtggatgtgcaaagcgacatcatcgatactcctggtcgtcgcacggtgattccgttggcttctgcacggttactgagcgacaaagtttcccgtgaactctatccggttgtccacattggggatgaaagctggcgtatgatgaccacggatatggcgtcagtaccagtgtcggtaattggcgaagaggttgcggatctgtcgcatcgcgagaatgacatcaagaacgccattaacctgatgttttggggcatttaattaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaccatgattacgccaagctatctgggcgataccattgaagactggagttcagacgtgtgctcttccgatctgat